(19) United States
(12) Patent Application Publication
(10) Pub. No.: US 2025/0049922 A1
(43) Pub. Date: Feb. 13, 2025

(54) PHARMACEUTICAL COMPOSITION COMPRISING P-BORONOPHENYLALANINE

(71) Applicant: Tenboron Oy, Helsinki (FI)

(72) Inventors: Tero Satomaa, Helsinki (FI); Ritva Niemela, Helsinki (FI); Juhani Saarinen, Helsinki (FI); Jari Helin, Rajamaki (FI)

(73) Assignee: Tenboron Oy, Helsinki (FI)

(21) Appl. No.: 18/717,577

(22) PCT Filed: Dec. 12, 2022
(86) PCT No.: PCT/FI2022/050826

§ 371 Date: Jun. 7, 2024

(30) Foreign Application Priority Data
Dec. 13, 2021 (FI) 20216268

(51) Int. Cl. A61K 41/00; A61K 47/18; A61K 47/26

(52) U.S. Cl. CPC A61K 41/0095; A61K 47/18; A61K 47/26

(57) ABSTRACT

A pharmaceutical composition is disclosed. The pharmaceutical composition may comprise boronophenylalanine (BPA) or a pharmaceutically acceptable salt thereof and a 2-hydroxy amine compound (2HA) or a pharmaceutically acceptable salt thereof.

### RELATED APPLICATIONS

This application is filed under 35 U.S.C. § 371 and claims priority to International Application No. PCT/FI2022/050826 filed on Dec. 12, 2022, and claims the benefit of Finland Application No. FI20216268 filed Dec. 13, 2021, which are hereby incorporated by reference in their entirety into this application.

### TECHNICAL FIELD

The present disclosure relates to pharmaceutical composition and to a method for producing the pharmaceutical composition.

### BACKGROUND

Boron neutron capture therapy (BNCT) is a form of noninvasive therapy of malignant tumors. In BNCT, a patient is injected with a drug which has the ability to localize in the tumor and which carries nonradioactive boron-10 atoms. When the drug is irradiated with low energy thermal neutrons, biologically destructive alpha particles and lithium-7 nuclei are emitted.

Drugs such as boronophenylalanine (hereinafter, also referred to as “BPA”) that are capable of localizing specifically in the tumor are required for BNCT. Such drugs should be easily produced, stable, soluble and safe. However, provision of such drugs is complicated e.g. by that some types of chemistries do not appear to work with boron-10 containing compounds.

In order to facilitate the administration of boron atoms to a patient and to facilitate the migration of the boron atoms to cancer cells, it is necessary to prepare an aqueous solution of an appropriate boron-containing compound. BPA has extremely poor aqueous solubility at physiological pH, while it can be solubilized with either strong acid or strong base. Various methods have been attempted to improve the solubility of BPA with respect to water. Among them, a method is known in which pH is increased by a base (sodium hydroxide or the like) to solubilize BPA, a saccharide (especially fructose) is added to form a BPA fructose complex, and the pH of the mixture is readjusted with an acid.

However, the water solubility of the BPA fructose complex may be insufficient. That is, for example, when the 30 g equivalent amount of BPA for a patient having a weight of 60 kg is prepared as a complex aqueous solution of fructose and fructose at room temperature, the volume of the solution may be at least about 1 L. Injection of such a large volume causes a large physical burden on the patient, and also it takes a long time to infuse the solution into the patient prior to neutron irradiation.

It would be desirable that a BPA formulation could also be freeze-dried and supplied to a medical institution as a powder. However, the freeze-dried powder of the BPA fructose complex has an extremely low dissolution rate to water at room temperature. Further, it is known that the stability of the BPA fructose complex is poor and that aqueous solutions of BPA and fructose have only a few days' shelf life.

There is therefore a need for pharmaceutical compositions that may have improved properties as compared to known BPA compositions and formulations.

### SUMMARY

A pharmaceutical composition is disclosed. The pharmaceutical composition may comprise boronophenylalanine (BPA) or a pharmaceutically acceptable salt thereof and a 2-hydroxy amine compound (2HA) or a pharmaceutically acceptable salt thereof, wherein the 2HA is selected from the compounds set forth in any one of the formulas I to II or a pharmaceutically acceptable salt thereof, or any combination or mixture thereof:

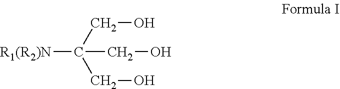

wherein R₁ and R₂ are each independently either H or selected from hydroxy-C₁-C₆-alkyl, 2-hydroxyethyl, 2,3,4,5,6-pentahydroxyhexyl, carboxy-C₁-C₆-alkyl, acetyl, C₁-C₆-alkyl, sulfo-C₁-C₆-alkyl, 3-sulfopropyl, 2-hydroxy-3-sulfopropyl, 1-sulfo-2-propanyl, 2-sulfoethyl, 3-[2-hydroxy-1,1-bis(hydroxymethyl)ethylamino]-C₁-C₆-alkyl, and 3-[2-hydroxy-1,1-bis(hydroxymethyl)ethylamino]propyl;

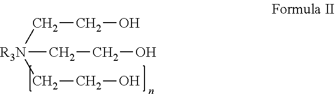

wherein n is 0 or 1;
provided that when n is 1, then R₃ is absent, and when n is 0, then R₃ is selected from H, hydroxy-C₁-C₆-alkyl, 2-hydroxyethyl, carboxy-C₁-C₆-alkyl, acetyl, C₁-C₆-alkyl, sulfo-C₁-C₆-alkyl and 2-sulfoethyl.

### BRIEF DESCRIPTION OF THE DRAWINGS

FIG. 1. Comparison of BPA-T and BPA-F biodistribution in tumor mice two hours after dosing. The graph shows box plots of ¹⁰B concentration in all sampled tissues as well as scatterplots for each individual mouse for BPA-F 30 g/L (F, n=6) and BPA-T 30 g/L (T, n=6). ¹⁰B concentration is on the y-axis (μg/kg=ppm, parts per million). Filled box shows the interquartile range (IQR) between the 25th percentile (Q1) and the 75th percentile (Q3). Whiskers show the minimum (Q1−1.5×IQR) and the maximum (Q3+1.5×IQR). Solid line inside the box shows the median and dashed line shows the mean. Muscle=cheek muscle. Saliv.gland=salivary glands.

FIG. 2. Tumor:tissue ¹⁰B ratios of BPA-T and BPA-F in tumor mice two hours after dosing. The graph shows box plots of tumor:tissue ¹⁰B ratios in all sampled tissues as well as scatterplots for each individual mouse for BPA-F 30 g/L (F, n=6) and BPA-T 30 g/L (T, n=6). Box plots are drawn as in FIG. 1. Muscle=cheek muscle. Saliv.gland=salivary glands.

FIG. 3. Time series of BPA-T and BPA-F between 1-8 hours after dosing. ¹⁰B concentrations decreased as a function of time similarly after both BPA-F and BPA-T administration in the tumor, cheek muscle and blood samples. Error bars show the standard deviation (STD). n=3 at each time point.

FIG. 4. Comparison of biodistribution at two hours after dosing of BPA-T (T, n=9), BPA-F (F, n=9), BPA-mannitol (M, n=6), BPA-sorbitol (S, n=6), all 30 g/L, and BPA-T 120 g/L (T120, n=6). The graph shows box plots of tumor:blood and tumor:muscle ¹⁰B ratios as well as scatterplots for each individual mouse. Box plots are drawn as in FIG. 1. Muscle=cheek muscle. Saliv.gland=salivary glands.

### DETAILED DESCRIPTION

A pharmaceutical composition is disclosed.

The pharmaceutical composition disclosed herein is suitable for use in boron neutron capture therapy.

In the context of this specification, the term “boron neutron capture therapy” (BNCT) may be understood as referring to targeted radiotherapy, wherein nonradioactive boron-10 is irradiated with low energy thermal neutrons to yield biologically destructive alpha particles and lithium-7 nuclei. The nonradioactive boron-10 may be targeted by incorporating it in a tumor localizing drug. Examples of such tumor localizing drugs are tumor localizing conjugates and boronophenylalanine.

The pharmaceutical composition may comprise boronophenylalanine (BPA) or a pharmaceutically acceptable salt thereof and a 2-hydroxy amine compound (2HA) or a pharmaceutically acceptable salt thereof.

In the context of this specification, the term “2-hydroxy amine compound” or “2HA” may be understood as referring to any 2-hydroxy amine compound disclosed in this specification, or one or more of the 2-hydroxy amine compounds disclosed in this specification. The 2HA may have an amine group and at least two hydroxyl groups in the β-position relative to the amine group. Unless explicitly indicated otherwise, the term “2-hydroxy amine compound” or “2HA” may also be understood as referring to a pharmaceutically acceptable salt of the 2HA. For example, 2HA compounds that are tertiary amines, may form pharmaceutically acceptable salts.

The amine group may be a primary, secondary or tertiary amine group.

In the context of this specification, unless explicitly indicated otherwise, the term “BPA” may also be understood as referring to a pharmaceutically acceptable salt of BPA.

Not to be bound by theory, it may be that the configuration of the hydroxyl groups and the amine group in the 2HA promotes the aqueous solubility of BPA. The 2HA may be capable of forming a complex with the boronate group of BPA via one or more of its hydroxyl groups and/or its amine group, thus promoting the aqueous solubility of BPA.

The 2HA may be selected from the compounds set forth in any one of the formulas I to II, pharmaceutically acceptable salts thereof, or any combination or mixture thereof:

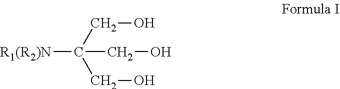

wherein R₁ and R₂ are each independently either H or selected from hydroxy-C₁-C₆-alkyl, 2-hydroxyethyl, 2,3,4,5,6-pentahydroxyhexyl, carboxy-C₁-C₆-alkyl, acetyl, C₁-C₆-alkyl, sulfo-C₁-C₆-alkyl, 3-sulfopropyl, 2-hydroxy-3-sulfopropyl, 1-sulfo-2-propanyl, 2-sulfoethyl, 3-[2-hydroxy-1,1-bis(hydroxymethyl)ethylamino]-C₁-C₆-alkyl, and 3-[2-hydroxy-1,1-bis(hydroxymethyl)ethylamino]propyl;

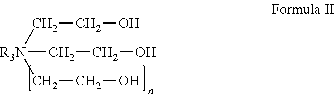

wherein n is 0 or 1;
provided that when n is 1, then R₃ is absent, and when n is 0, then R₃ is selected from H, hydroxy-C₁-C₆-alkyl, 2-hydroxyethyl, carboxy-C₁-C₆-alkyl, acetyl, C₁-C₆-alkyl, sulfo-C₁-C₆-alkyl and 2-sulfoethyl.

The 2HA may be selected from the compounds set forth in any one of the formulas I to II, pharmaceutically acceptable salts thereof, or any combination or mixture thereof:

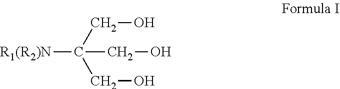

wherein R₁ and R₂ are each independently either H or selected from hydroxy-C₁-C₆-alkyl, 2,3,4,5,6-pentahydroxyhexyl, carboxy-C₁-C₆-alkyl, acetyl, C₁-C₆-alkyl, sulfo-C₁-C₆-alkyl, 2-hydroxy-3-sulfopropyl, and 3-[2-hydroxy-1,1-bis(hydroxymethyl)ethylamino]-C₁-C₆-alkyl;

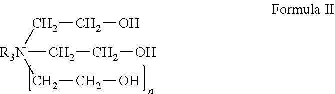

wherein n is 0 or 1;
provided that when n is 1, then R₃ is absent, and when n is 0, then R₃ is selected from H, hydroxy-C₁-C₆-alkyl, carboxy-C₁-C₆-alkyl, acetyl, C₁-C₆-alkyl, and sulfo-C₁-C₆-alkyl.

The hydroxy-C₁-C₆-alkyl may be e.g. a 2-hydroxyethyl.

The sulfo-C₁-C₆-alkyl may be e.g. a 3-sulfopropyl, 1-sulfo-2-propanyl, or 2-sulfoethyl.

The 3-[2-hydroxy-1,1-bis(hydroxymethyl)ethylamino]-C₁-C₆-alkyl may be e.g. 3-[2-hydroxy-1,1-bis(hydroxymethyl)ethylamino]propyl.

In the context of this specification, the term “BPA” may be understood as referring to boronophenylalanine, including any isomer(s) thereof. The term “BPA” may encompass any isomer of boronophenylalanine or a pharmaceutically acceptable salt thereof. BPA may be selected from the group consisting of p-boronophenylalanine, L-p-boronophenylalanine, D-p-boronophenylalanine, m-boronophenylalanine, L-m-boronophenylalanine, D-m-boronophenylalanine, o-boronophenylalanine, L-o-boronophenylalanine, D-o-boronophenylalanine, and any combination or mixture thereof. BPA may be selected from the group consisting of p-boronophenylalanine, L-p-boronophenylalanine, D-p-boronophenylalanine, m-boronophenylalanine, L-m-boronophenylalanine, D-m-boronophenylalanine, and any combination or mixture thereof.

The BPA may comprise or be L-p-boronophenylalanine or a pharmaceutically acceptable salt thereof.

L-p-boronophenylalanine has the following structure:

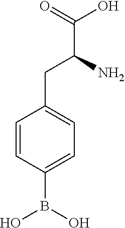 [SMILES: N[C@@H](Cc1ccc(B(O)O)cc1)C(=O)O]

BPA may, additionally or alternatively, comprise or be D-p-boronophenylalanine or a mixture of L-p-boronophenylalanine and D-p-boronophenylalanine, or a pharmaceutically acceptable salt thereof.

BPA may, additionally or alternatively, comprise or be m-boronophenylalanine (3-boronophenylalanine) or a mixture of p-boronophenylalanine and m-boronophenylalanine, or a pharmaceutically acceptable salt thereof.

BPA may, additionally or alternatively, comprise or be D-m-boronophenylalanine (3-boronophenylalanine) or a mixture of p-boronophenylalanine and m-boronophenylalanine, or a pharmaceutically acceptable salt thereof.

BPA may, additionally or alternatively, comprise or be L-m-boronophenylalanine (3-boronophenylalanine) or a mixture of p-boronophenylalanine and m-boronophenylalanine, or a pharmaceutically acceptable salt thereof.

L-m-boronophenylalanine has the following structure:

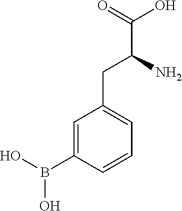 [SMILES: N[C@@H](Cc1cccc(B(O)O)c1)C(=O)O]

Alternatively, fluorine-18-BPA, i.e. [¹⁸F]BPA, may be used. The pharmaceutical composition may comprise [¹⁸F]BPA instead of or in addition to BPA. The BPA may be or comprise fluorine-18-BPA, i.e. [¹⁸F]BPA. The [¹⁸F]BPA is useful for imaging of a tumor with positron emission tomography (PET), which can be performed prior to BNCT. In an embodiment, the [¹⁸F]BPA is 4-borono-2-¹⁸F-fluoro-L-phenylalanine.

The BPA may have a normal isotopic distribution of about 20% of boron-10 and 80% of boron-11, or the BPA may be enriched with regard to boron-10. For BNCT, boron-10 enriched BPA may be preferred. In an embodiment, the BPA has at least 30%, at least 40%, at least 50%, at least 60%, at least 70%, at least 80%, at least 90%, at least 95%, at least 97%, at least 98%, or at least 99% boron-10. In this context, the percentage value may be understood as the percentage of boron-10 atoms of all boron atoms in the BPA. In an embodiment, the BPA is essentially pure with regard to boron-10. The BPA may be considered essentially pure with regard to boron-10 if at least 99.5% or about 100% of the boron atoms of the BPA are boron-10.

It has now been found that BPA can be dissolved at a high concentration in an aqueous solution of the 2-hydroxy amine compound (2HA), and the BPA remains in a state dissolved even at a relatively high concentration, also when pH is adjusted to a substantially physiological pH by adding an acid to the aqueous solution. It may be possible to obtain a dry formulation, such as a powder, of the pharmaceutical composition by drying the aqueous solution. Further, a powder obtained by freeze-drying the neutralized aqueous solution may be easily dissolved when mixed with water to give an aqueous solution containing BPA at the original (high) concentration.

In an embodiment, in the aqueous (dissolved) solution the BPA is distributed uniformly throughout the solution. In other words, the aqueous solution is a homogeneous solution; the BPA is dissolved in the aqueous solution such that the solution is completely uniform throughout. In an embodiment, the dissolved solution of BPA can be filtered without a change in BPA concentration and safely administered to the patient. In an embodiment, the BPA dissolved in the aqueous (dissolved) solution of BPA is capable of effectively localizing to a tumor, does not accumulate in normal tissues (at least in undesirable amounts, or accumulates less in normal tissues than in the tumor), and produces favourable tumor:tissue and tumor:blood gradients for safe and effective administration of neutron radiation to the patient.

In an embodiment, BPA may be considered to be dissolved when at least 95%, at least 97%, at least 98%, at least 99%, at least 99.5% or essentially 100% of the BPA is in the dissoluted (dissolved) phase. The dissolution of BPA may be analyzed with various methods known in the art. For example, non-dissolved BPA can be filtered or centrifuged out of the solution, after which the proportion of the BPA (dissolution %) in the solution phase can be analyzed by for example UV (ultraviolet) spectrophotometry, infrared spectroscopy, NMR (nuclear magnetic resonance) spectroscopy or chromatography (for suitable analysis methods see for example Pierro et al. 2000, Anal. Biochem. 284:301-6 and Heikkinen et al. 2011, J. Radiat. Res. 52:360-4), or by direct measurement of boron content (for example by ICP-MS as described in Verlinden et al. 2021, J. Anal. At. Spectrom. 36:598-606). In an embodiment, the BPA may be considered dissolved when the aqueous solution is clear upon visual inspection.

The 2HA may be selected from the compounds set forth in formula I, pharmaceutically acceptable salts thereof, or any combination or mixture thereof:

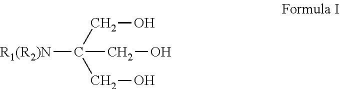

wherein R₁ and R₂ are each independently either H or selected from hydroxy-C₁-C₆-alkyl, 2-hydroxyethyl, 2,3,4,5,6-pentahydroxyhexyl, carboxy-C₁-C₆-alkyl, acetyl, C₁-C₆-alkyl, sulfo-C₁-C₆-alkyl, 3-sulfopropyl, 2-hydroxy-3-sulfopropyl, 1-sulfo-2-propanyl, 2-sulfoethyl, 3-[2-hydroxy-1,1-bis(hydroxymethyl)ethylamino]-C₁-C₆-alkyl, and 3-[2-hydroxy-1,1-bis(hydroxymethyl)ethylamino]propyl.

The 2HA may be selected from the compounds set forth in formula II, pharmaceutically acceptable salts thereof, or any combination or mixture thereof:

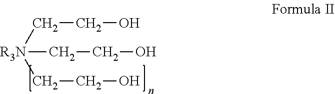

wherein n is 0 or 1; and
when n=1, then R₃ is absent, and when n=0, then R3 is selected from H, hydroxy-C₁-C₆-alkyl, 2-hydroxyethyl, carboxy-C₁-C₆-alkyl, acetyl, C₁-C₆-alkyl, sulfo-C₁-C₆-alkyl and 2-sulfoethyl.

The 2HA may be selected from the following compounds, pharmaceutically acceptable salts, and any mixtures and combinations thereof:
Tris(hydroxymethyl)aminomethane (Tris),
2-[Bis(2-hydroxyethyl)amino]-2-(hydroxymethyl)propane-1,3-diol (Bis-Tris),
(2-hydroxyethyl)amino-tris(hydroxymethyl) methane,
N-(Tris(hydroxymethyl)methyl)glycine (Tricine),
2-(Dimethylamino)-2-(hydroxymethyl) propane-1,3-diol (N,N-dimethyl-Tris),
1-Deoxy-1-{[1,3-dihydroxy-2-(hydroxymethyl)-2-propanyl]amino}hexitol,
2-{[1,3-Dihydroxy-2-(hydroxymethyl)-2-propanyl]amino}-1-propanesulfonic acid,
[Tris(hydroxymethyl)methylamino]propanesulfonic acid (TAPS),
3-[N-Tris(hydroxymethyl)methylamino]-2-hydroxypropanesulfonic acid (TAPSO),
2-{[1,3-Dihydroxy-2-(hydroxymethyl)propan-2-yl]amino}ethane-1-sulfonic acid (TES),
Bis-Tris propane (BTP),
Diethanolamine (DEA),
Triethanolamine (TEA),
[Bis(2-hydroxyethyl)amino]acetic acid (Bicine), and
N,N-Bis(2-hydroxyethyl)-2-aminoethanesulfonic acid (BES).

The 2HA may be selected from compounds according to any one of Formulas Ia-IId, pharmaceutically acceptable salts thereof, any mixtures and combinations thereof. Tris(hydroxymethyl)aminomethane (Tris, also called as TRIS, Tris base, Trizma, Trisamine, TRAM, Tromethamine, Trometamol, Tromethane or Trisaminol) according to Formula Ia:

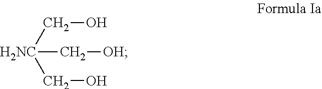

2-[Bis(2-hydroxyethyl)amino]-2-(hydroxymethyl)propane-1,3-diol (Bis-Tris) according to Formula Ib:

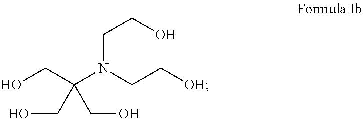 [SMILES: OCCN(CCO)C(CO)(CO)CO]

(2-hydroxyethyl)amino-tris(hydroxymethyl)methane according to Formula Ic:

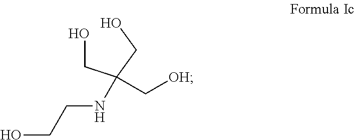 [SMILES: OCCNC(CO)(CO)CO]

N-(Tris(hydroxymethyl)methyl)glycine (Tricine) according to Formula Id:

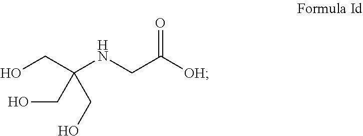 [SMILES: O=C(O)CNC(CO)(CO)CO]

2-(Dimethylamino)-2-(hydroxymethyl)propane-1,3-diol (N,N-dimethyl-Tris) according to Formula Ie:

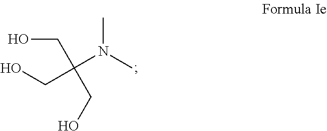

1-Deoxy-1-{[1,3-dihydroxy-2-(hydroxymethyl)-2-propanyl]amino}hexitol according to Formula If:

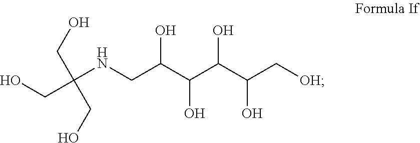 [SMILES: OCC(O)C(O)C(O)C(O)CNC(CO)(CO)CO]

2-{[1,3-Dihydroxy-2-(hydroxymethyl)-2-propanyl]amino}-1-propanesulfonic acid according to Formula Ig:

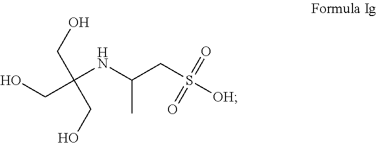

[Tris(hydroxymethyl)methylamino]propanesulfonic acid (TAPS) according to Formula Ih:

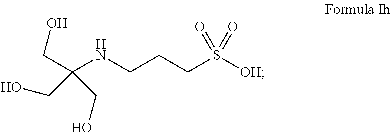 [SMILES: O=S(=O)(O)CCCNC(CO)(CO)CO]

3-[N-Tris(hydroxymethyl)methylamino]-2-hydroxypropanesulfonic acid (TAPSO) according to Formula II:

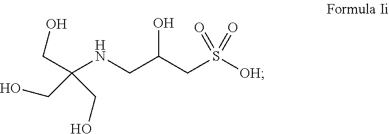 [SMILES: O=S(=O)(O)CC(O)CNC(CO)(CO)CO]

2-{[1,3-Dihydroxy-2-(hydroxymethyl) propan-2-yl]amino}ethane-1-sulfonic acid TES according to Formula Ij:

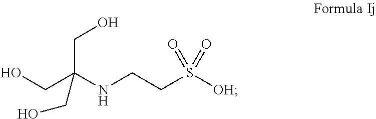 [SMILES: O=S(=O)(O)CCNC(CO)(CO)CO]

Bis-Tris propane (BTP) according to Formula Ik:

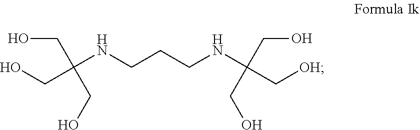 [SMILES: OCC(CO)(CO)NCCCNC(CO)(CO)CO]

Diethanolamine (DEA) according to Formula IIa:

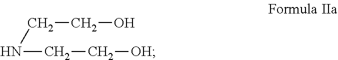

Triethanolamine (TEA) according to Formula IIb:

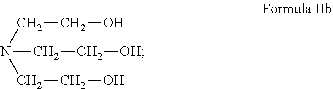

[Bis(2-hydroxyethyl)amino]acetic acid (Bicine) according to Formula IIc:

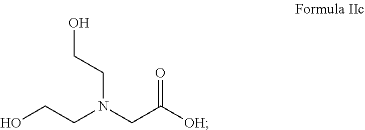 [SMILES: O=C(O)CN(CCO)CCO]

N,N-Bis(2-hydroxyethyl)-2-aminoethanesulfonic acid (BES) according to Formula IId:

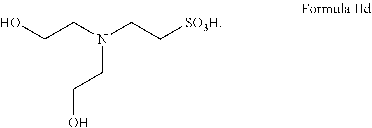 [SMILES: O=S(=O)(O)CCN(CCO)CCO]

The 2HA may comprise or be Tris(hydroxymethyl)aminomethane (Tris) or a pharmaceutically acceptable salt thereof. Tris(hydroxymethyl)aminomethane (Tris) is represented by Formula Ia:

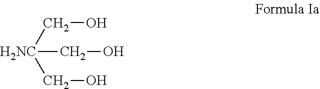

The 2HA may comprise or be 2-[Bis(2-hydroxyethyl)amino]-2-(hydroxymethyl)propane-1,3-diol (Bis-Tris) or a pharmaceutically acceptable salt thereof. 2-[Bis(2-hydroxyethyl)amino]-2-(hydroxymethyl)propane-1,3-diol (Bis-Tris) is represented by Formula Ib:

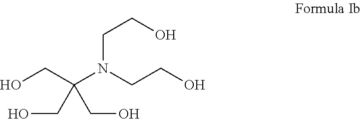 [SMILES: OCCN(CCO)C(CO)(CO)CO]

The 2HA may comprise or be (2-hydroxyethyl)amino-tris(hydroxymethyl)methane or a pharmaceutically acceptable salt thereof. (2-Hydroxyethyl)amino-tris(hydroxymethyl)methane is represented by Formula Ic:

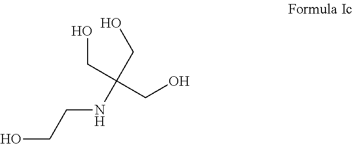 [SMILES: OCCNC(CO)(CO)CO]

The 2HA may comprise or be N-(Tris(hydroxymethyl)methyl)glycine (Tricine), i.e. {[1,3-dihydroxy-2-(hydroxymethyl)propan-2-yl]amino}acetic acid, or a pharmaceutically acceptable salt thereof. N-(Tris(hydroxymethyl)methyl)glycine (Tricine) is represented by Formula Id:

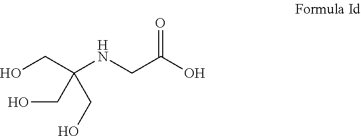 [SMILES: O=C(O)CNC(CO)(CO)CO]

The 2HA may comprise or be 2-(Dimethylamino)-2-(hydroxymethyl) propane-1,3-diol (N,N-dimethyl-Tris) or a pharmaceutically acceptable salt thereof. 2-(Dimethylamino)-2-(hydroxymethyl)propane-1,3-diol (N,N-dimethyl-Tris) is represented by Formula Ie:

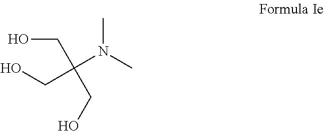

The 2HA may comprise or be 1-Deoxy-1-{[1,3-dihydroxy-2-(hydroxymethyl)-2-propanyl]amino}hexitol or a pharmaceutically acceptable salt thereof. 1-Deoxy-1-{[1,3-dihydroxy-2-(hydroxymethyl)-2-propanyl]amino}hexitol is represented by Formula If:

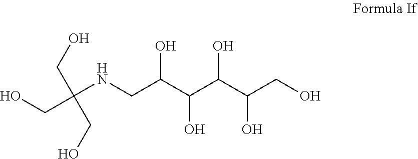 [SMILES: OCC(O)C(O)C(O)C(O)CNC(CO)(CO)CO]

The 2HA may comprise or be 2-{[1,3-Dihydroxy-2-(hydroxymethyl)-2-propanyl]amino}-1-propanesulfonic acid or a pharmaceutically acceptable salt thereof. 2-{[1,3-Dihydroxy-2-(hydroxymethyl)-2-propanyl]amino}-1-propanesulfonic acid is represented by Formula Ig:

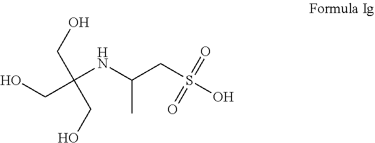

The 2HA may comprise or be [Tris(hydroxymethyl)methylamino]propanesulfonic acid (TAPS) or a pharmaceutically acceptable salt thereof. [Tris(hydroxymethyl)methylamino]propanesulfonic acid (TAPS) is represented by Formula Ih:

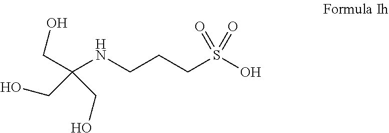 [SMILES: O=S(=O)(O)CCCNC(CO)(CO)CO]

The 2HA may comprise or be 3-[N-Tris(hydroxymethyl)methylamino]-2-hydroxypropanesulfonic acid (TAPSO) or a pharmaceutically acceptable salt thereof. 3-[N-Tris(hydroxymethyl)methylamino]-2-hydroxypropanesulfonic acid (TAPSO) is represented by Formula Ii:

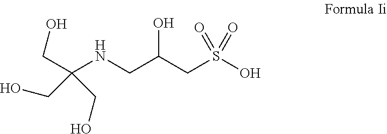 [SMILES: O=S(=O)(O)CC(O)CNC(CO)(CO)CO]

The 2HA may comprise or be 2-{[1,3-Dihydroxy-2-(hydroxymethyl)propan-2-yl]amino}ethane-1-sulfonic acid (TES) or a pharmaceutically acceptable salt thereof. 2-{[1,3-Dihydroxy-2-(hydroxymethyl)propan-2-yl]amino}ethane-1-sulfonic acid (TES) is represented by Formula Ij:

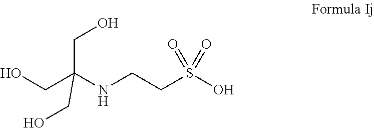 [SMILES: O=S(=O)(O)CCNC(CO)(CO)CO]

The 2HA may comprise or be Bis-Tris propane (BTP) or a pharmaceutically acceptable salt thereof. Bis-Tris propane (BTP) is represented by Formula Ik:

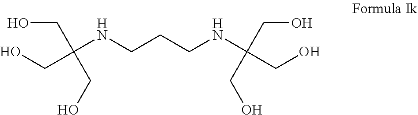 [SMILES: OCC(CO)(CO)NCCCNC(CO)(CO)CO]

The 2HA may comprise or be diethanolamine (DEA) or a pharmaceutically acceptable salt thereof. Diethanolamine is represented by Formula IIa:

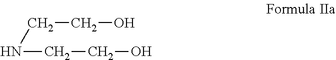

The 2HA may comprise or be triethanolamine (TEA) or a pharmaceutically acceptable salt thereof. Triethanolamine is represented b Formula IIb:

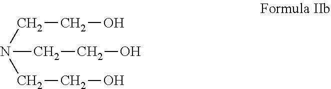

The 2HA may comprise or be [Bis(2-hydroxyethyl)amino]acetic acid (Bicine) or a pharmaceutically acceptable salt thereof. [Bis(2-hydroxyethyl)amino]acetic acid (Bicine) is represented by Formula IIc:

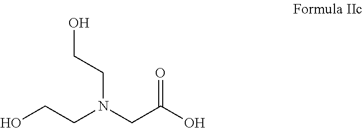 [SMILES: O=C(O)CN(CCO)CCO]

The 2HA may comprise or be N,N-Bis(2-hydroxyethyl)-2-aminoethanesulfonic acid (BES) or a pharmaceutically acceptable salt thereof. N,N-Bis(2-hydroxyethyl)-2-aminoethanesulfonic acid (BES) is represented by Formula IId:

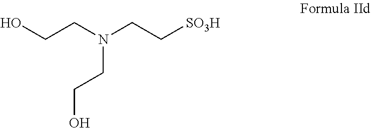 [SMILES: O=S(=O)(O)CCN(CCO)CCO]

The molar ratio of 2HA:BPA in the pharmaceutical composition may be in the range of 0.5-3, about 0.5, in the range of 0.5-1, about 1, in the range of 1-1.5, in the range of 1-2, about 2, in the range of 2-3, or about 3.

When the molar ratio of 2HA:BPA is at least 1, the dissolution of BPA may be improved.

In embodiments in which Tris is the 2HA, the molar ratio of Tris:BPA in the pharmaceutical composition may be in the range of 0.5-3, about 0.5, in the range of 0.5-1, about 1, in the range of 1-1.5, in the range of 1-2, about 2, in the range of 2-3, or about 3.

In embodiments in which Tris is the 2HA and L-p-boronophenylalanine is the BPA, the molar ratio of Tris(hydroxymethyl)aminomethane(Tris):L-p-boronophenylalanine may be in the range of 0.5-3, about 0.5, in the range of 0.5-1, about 1, in the range of 1-1.5, in the range of 1-2, about 2, in the range of 2-3, or about 3.

The pharmaceutical composition may be an aqueous solution. In other words, the pharmaceutical composition may be in the form of an aqueous solution. The BPA (and the 2HA) may thus be dissolved in the aqueous solution.

The aqueous solution may have a pH in the range of 6.5 to 8.5.

The aqueous solution may have a pH in the range of 7 to 8, in the range of 7.3 to 7.5, in the range of 7.35 to 7.45, about pH 7.4, or a physiological pH or a substantially physiological pH.

In the context of this specification, the term “substantially neutral pH” may be understood as referring to a pH in the range of about pH 6.5 to 7.5, or about 7.

In the context of this specification, the term “substantially physiological pH” may be understood as referring to a pH in the range of about 6.5 to 8.5. In the context of this specification, the term “physiological pH” may be understood as referring to a pH in the range of about 7.35-7.45, or about pH 7.4.

The concentration of BPA in the aqueous solution may be at least 30 g/L.

The BPA concentration in the aqueous solution is not particularly limited and can be set as appropriate. However, in general, it may be desirable to have a higher concentration than the conventional BPA fructose complex aqueous solution of about 30 g/L. This is because the volume of the aqueous solution to be administered to the patient may then be reduced (as compared to the volume of the BPA fructose complex aqueous solution). Therefore, the BPA concentration may be at least 30 g/L, or above 30 g/L, above 35 g/L, above 40 g/L, above 45 g/L, above 50 g/L, above 55 g/L, or above 60 g/L, for example. The concentration of BPA in the aqueous solution may be in the range of 30-300 g/L, or in the range of 30-100 g/L, or in the range of 100-300 mg/ml, or in the range of 30-60 g/L, or in the range of 30-45 g/L, or about 30 g/L, or 30 g/L.

The aqueous solution may be isotonic or substantially isotonic.

The aqueous solution may be hypertonic.

In an embodiment, the osmolality of the aqueous solution is such that the aqueous solution is suitable for intravenous administration to a subject.

In an embodiment, the aqueous solution has an osmolality of 310 mOsm/L or greater, or of 320 mOsm/L or greater. In an embodiment, the aqueous solution has an osmolality in the range of 250-350 mOsm/L, or in the range of 280-330 mOsm/L, or in the range of about 300-310 mOsm/L.

In an embodiment, the aqueous solution has an osmolality in the range of 310-900 mOsm/L, in the range of 310-600 mOsm/L, in the range of 310-500 mOsm/L, in the range of 310-400 mOsm/L, in the range of 400-600 mOsm/L, in the range of 400-500 mOsm/L, or in the range of 500-600 mOsm/L. In an embodiment, the aqueous solution has an osmolality of 900 mOsm/L or lower, of 800 mOsm/L or lower, of 700 mOsm/L or lower, of 600 mOsm/L or lower, or 500 mOsm/L or lower, or of 400 mOsm/L or lower. Relatively high osmolalities may not be well suited for intravenous administration.

The pharmaceutical composition may be a dry formulation. In other words, the pharmaceutical composition may be in the form of a dry formulation. For example, the dry formulation may be a powder.

The dry formulation, for example a powder, may be obtainable by drying, e.g. by freeze-drying the aqueous solution. The dry formulation may be easily dissolved when mixed with water. Upon addition of water, i.e. when mixed with water, the dry formulation may give an aqueous solution containing BPA at the original (high) concentration.

The dry formulation may be capable of forming an aqueous solution having a pH in the range of 6.5 to 8.5 upon addition of water. Upon the addition of water, the dry formulation may dissolve, thereby forming the aqueous solution having a pH in the range of 6.5 to 8.5. For example, the dry formulation may be capable of forming an aqueous solution having a pH in the range of 6.5 to 8.5 upon addition of 1 L of (pure, for example distilled or deionized) water to a dry formulation comprising at least 30 g, or 30 g, of BPA.

The dry formulation may be capable of forming an aqueous solution having a pH in the range of 7 to 8, in the range of 7.3 to 7.5, in the range of 7.35 to 7.45, about pH 7.4, or a physiological pH or a substantially physiological pH.

In the context of this specification, the term “water” may be understood as referring to e.g. distilled water, deionized water, or other form of water considered as pure water. However, additionally or alternatively, it may refer e.g. to a saline solution, a buffer solution, a buffered saline solution, or other aqueous solution comprising water.

Since the aqueous solution may be administered to a subject by mixing with an infusion solution, it is not always necessary to strictly adjust the pH to a neutral or physiological pH. This is because the buffer effect of the buffer contained in the infusion solution may compensate for any deviation from a neutral or physiological pH.

The aqueous solution of BPA and 2HA has a high stability in essentially physiological pH and is almost completely stabile in substantially neutral pH. Therefore, the pH of the aqueous solution may be appropriately set so as to be convenient for use as a product in e.g. a substantially neutral pH, slightly alkaline pH, or essentially physiological pH.

The pharmaceutical composition may further comprise additional components or additives.

In other words, the pharmaceutical composition does not necessarily exclude components other than BPA, water and 2HA, unless such components would substantially adversely affect its stability (solubility and storage stability). It may be naturally assumed that the conjugated base (in the case of hydrochloric acid, Cl⁻ ion) of an acid to be added at the time of pH adjustment is contained in the pharmaceutical composition. Other than the above, a component derived from an agent for adjusting pH, such as sodium hydrogen carbonate (which can be used for fine adjustment at the time of preparation of the pharmaceutical composition and adjusting its pH), may be included. The agent for adjusting the pH of the pharmaceutical composition may be e.g. an acid. The agent for adjusting the pH of the pharmaceutical composition may be e.g. an alkali, e.g. sodium hydroxide, potassium hydroxide and/or sodium carbonate.

The pharmaceutical composition may further comprise at least one of hydrochloric acid, sodium chloride, acetic acid, sodium acetate, polyethylene glycol, a polyol, a saccharide, fructose, mannitol or sorbitol.

The pharmaceutical composition may further comprise at least one of sodium hydroxide, potassium hydroxide or sodium carbonate.

In an embodiment, the pharmaceutical composition does not comprise or is free of polyethylene glycol, a polyol, a saccharide, fructose, mannitol or sorbitol.

In an embodiment, the pharmaceutical composition comprises a polyol.

In the context of this specification, the term “polyol” may be understood as referring to an organic compound containing multiple hydroxyl groups. In an embodiment, the polyol is selected from the group of a diol, a triol, a tetrol, a pentol and a hexol. In an embodiment, the polyol is a reduced saccharide, such as sorbitol, mannitol, and the like. In an embodiment, the polyol is a non-reducing saccharide, such as saccharose, trehalose, and the like. In the context of this specification, “a polyol” or “polyol” may refer to one or more polyols. In other words, the polyol may comprise or be a polyol or a mixture of two or more polyols.

The polyol may comprise or be mannitol. Mannitol is a polyol well suited for the pharmaceutical composition.

In an embodiment, the pharmaceutical composition consists of BPA, the 2HA and water.

In an embodiment, the pharmaceutical composition comprises or consists of BPA, the 2HA, a polyol and water. In an embodiment, the molar proportions of the BPA, the 2HA, and the polyol are such that the sum of the molar amounts of the 2HA and the polyol is larger than the molar amount of BPA, so that [2HA]+[polyol]>[BPA]. The molar proportions may be understood such that [2HA] is the molar amount of the 2HA, [polyol] is the molar amount of the polyol (i.e. the (combined) molar amount of the one or more polyols), and [BPA] is the molar amount of the BPA. In other words, if the polyol comprises or is a mixture of two or more different polyols, then [polyol] is the sum of the molar amounts of the two or more different polyols. As an example, if [BPA] is 1 mol, then [2HA]+[polyol]>1 mol.

The term “molar amount” may be understood as referring to the number of moles, i.e. the number of moles in the pharmaceutical composition.

In an embodiment, the molar proportions and/or molar amounts of the BPA, the 2HA, and the polyol are such that the sum of the molar amounts of BPA and polyol is about equal to or larger than the molar amount of BPA.

In the embodiments herein, [2HA] is the molar amount of the 2HA, [polyol] is the molar amount of the polyol, [BPA] is the molar amount of the BPA, and x is the multiplication sign. In other words, 3×[BPA] may be understood as referring to three times the molar amount of the BPA. If the polyol comprises or is a mixture of two or more different polyols, then [polyol] is the sum of the molar amounts of the two or more different polyols.

In an embodiment, [2HA]+[polyol]>1.5×[BPA]. In an embodiment, [2HA]+[polyol]>2×[BPA]. In an embodiment, [BPA]<[2HA]+[polyol]<3×[BPA]. In an embodiment, [BPA]<[2HA]+[polyol]<2.5×[BPA]. In an embodiment, [BPA]<[2HA]+[polyol]<2×[BPA].

In an embodiment, [2HA]+[polyol]≥0.8×[BPA]. In an embodiment, [2HA]+[polyol]≥0.9×[BPA]. In an embodiment, [2HA]+[polyol]≥1.0×[BPA]. In an embodiment, [2HA]+[polyol]≥1.5×[BPA]. In an embodiment, [2HA]+[polyol]≥2.0×[BPA].

In an embodiment, 0.8×[BPA]≤[2HA]+[polyol]≤5.0×[BPA]. In an embodiment, 0.9×[BPA]≤[2HA]+[polyol]≤4.0×[BPA]. In an embodiment, 1.0×[BPA]≤[2HA]+[polyol]≤3.0×[BPA]. In an embodiment, 1.5×[BPA]≤[2HA]+[polyol]≤2.5×[BPA]. In an embodiment, 2.0×[BPA]≤[2HA]+[polyol]≤2.2×[BPA].

In an embodiment, [2HA]+[polyol]=2.1×[BPA].

In an embodiment, the pharmaceutical composition comprises or consists of BPA, the 2HA, water and a component derived from an agent for adjusting the pH of the pharmaceutical composition.

In an embodiment, the pharmaceutical composition comprises or consists of BPA, the 2HA, a polyol, water and a component derived from an agent for adjusting the pH of the pharmaceutical composition.

In an embodiment, the pharmaceutical composition comprises L-p-boronophenylalanine. In other words, the BPA may be L-p-boronophenylalanine.

In an embodiment, the pharmaceutical composition comprises Tris(hydroxymethyl)aminomethane (Tris). In other words, the 2HA may be Tris(hydroxymethyl)aminomethane.

In an embodiment, the pharmaceutical composition comprises mannitol. In other words, the polyol may be mannitol.

In an embodiment, the pharmaceutical composition comprises L-p-boronophenylalanine as the BPA, Tris(hydroxymethyl)aminomethane (Tris) as the 2HA, and mannitol as the polyol.

In an embodiment, the pharmaceutical composition comprises L-p-boronophenylalanine, Tris(hydroxymethyl)aminomethane (Tris), and mannitol.

The molar ratio of Tris(hydroxymethyl)aminomethane (Tris):L-p-boronophenylalanine may be in the range of 0.5-3, about 0.5, in the range of 0.5-1, about 1, in the range of 1-1.5, in the range of 1-2, about 2, in the range of 2-3, or about 3.

In the embodiments below, [Tris] is the molar amount of Tris, [mannitol] is the molar amount of mannitol, and [BPA] is the molar amount of L-p-boronophenylalanine.

In an embodiment, [Tris]+[mannitol]>[L-p-boronophenylalanine], wherein [Tris] is the molar amount of Tris, [mannitol] is the molar amount of mannitol, and [L-p-boronophenylalanine] is the molar amount of L-p-boronophenylalanine. In an embodiment, [Tris]+[mannitol]>1.5×[L-p-boronophenylalanine]. In an embodiment, [Tris]+[mannitol]>2×[L-p-boronophenylalanine]. In an embodiment, [Tris]+[mannitol]<3×[L-p-boronophenylalanine]. In an embodiment, [Tris]+[mannitol]<2.5×[L-p-boronophenylalanine]. In an embodiment, [Tris]+[mannitol]<2×[L-p-boronophenylalanine].

In an embodiment, the pharmaceutical composition comprises L-p-boronophenylalanine, Tris and mannitol in molar proportions 10:10:11.

In an embodiment, the pharmaceutical composition comprises L-p-boronophenylalanine, Tris and mannitol in molar proportions 10:10:11, and the pH of the composition is between pH 6.5-7.5, or about pH 7.4 or a physiological pH or a substantially physiological pH. In an embodiment, [Tris]+[mannitol]≥0.8×[BPA]. In an embodiment, [Tris]+[mannitol]≥0.9×[BPA]. In an embodiment, [Tris]+[mannitol]≥1.0×[BPA]. In an embodiment, [Tris]+[mannitol]≥1.5×[BPA]. In an embodiment, [Tris]+[mannitol]≥2.0×[BPA].

In an embodiment, 0.8×[BPA]≤[Tris]+[mannitol]≤5.0×[BPA]. In an embodiment, 0.9×[BPA]≤[Tris]+[mannitol]≤4.0×[BPA]. In an embodiment, 1.0×[BPA]≤[Tris]+[mannitol]≤3.0×[BPA]. In an embodiment, 1.5×[BPA]≤[Tris]+[mannitol]≤2.5×[BPA]. In an embodiment, 2.0×[BPA]≤[Tris]+[mannitol]≤2.2×[BPA].

In an embodiment, [Tris]+[mannitol]=2.1×[BPA].

In the above embodiments indicating the molar ratios and/or molar amounts, the pharmaceutical composition may be in the form of an aqueous solution or a dry formulation.

The pharmaceutical composition may be supplied as a product in the form of an aqueous solution. Alternatively or additionally, the pharmaceutical composition may be supplied as a solid preparation, i.e. dry composition. In such a solid preparation, the aqueous solution is dried. The dried product may be sealed in a packaging for melting. Although it may be convenient to use freeze-drying in the drying of the aqueous solution, the drying method is not particularly limited. The drying may be done using a method in which a sterile formulation suitable for injection can be supplied.

The pharmaceutical composition may have a high stability with time even in the form of a freeze-dried product and can be easily restored to the form of an aqueous solution by adding e.g. distilled water for injection before use.

The pharmaceutical composition may be stable over a time period of up to 24 months, or up to 12 months, or at least 1 month, or at least 2 years or at least 3 years.

The pharmaceutical composition may be stable over such a time period at a temperature of −90-+40° C. In an embodiment, the pharmaceutical composition may be stable over such a time period at a temperature of −90 to 0° C., −60 to 0° C., −30 to 0° C., 0 to 10° C., 2 to 8° C., 2 to 6° C., 0 to 30° C., or 10 to 25° C. For example, the pharmaceutical composition may be stable over a time period of up to 24 months, or up to 12 months, or at least 1 month, or at least 2 years or at least 3 years at a temperature of 2 to 6° C. In an embodiment, the pharmaceutical composition may be stable over a time period of up to 24 months, or up to 12 months, or at least 1 month, or at least 2 years or at least 3 years at normal room temperature of 20 to 24° C. The pharmaceutical composition may be considered stable when it remains clear and there is no precipitation, or when after dissolution it remains clear and there is no precipitation.

It was found that the main degradation route of the BPA comprised in the pharmaceutical composition may be the formation of phenylalanine (Phe) from BPA. The pharmaceutical composition may be considered stable when after storage (for a period of time, for example up to 24 months, or up to 12 months, or at least 1 month, or at least 2 years or at least 3 years) the amount of Phe formed during storage, by weight %, is less than about 5 w-%, less than 4 w-%, less than 3 w-%, less than 2 w-%, less than 1 w-%, less than 0.5 w-%, less than 0.2 w-%, less than 0.1 w-% or about 0 w-% of the total amount of BPA and Phe in the pharmaceutical composition. The amounts of Phe and BPA may be determined by for example RP-HPLC or another method in the art.

The pharmaceutical composition according to one or more embodiments described in this specification for use in therapy is disclosed.

The pharmaceutical composition according to one or more embodiments described in this specification for use in the treatment of cancer is disclosed.

The cancer may be a head-and-neck cancer.

The cancer may be selected from the group consisting of head-and-neck cancer, brain cancer, glioma, skin cancer, melanoma, leukemia, lymphoma, breast cancer, prostate cancer, ovarian cancer, colorectal cancer, gastric cancer, squamous cancer, small-cell lung cancer, multidrug resistant cancer and testicular cancer.

The pharmaceutical composition according to one or more embodiments described in this specification for use in boron neutron capture therapy (BNCT) is disclosed.

The BPA may be or comprise fluorine-18-BPA, i.e. [¹⁸F]BPA, such as 4-borono-2-¹⁸F-fluoro-L-phenylalanine. In such embodiments, the pharmaceutical composition may be administered to a subject having a tumor; the tumor may be imaged with positron emission tomography (PET); and subsequently the subject may be treated by BNCT. In an embodiment, the [¹⁸F]BPA is 4-borono-2-¹⁸F-fluoro-L-phenylalanine.

Use of the pharmaceutical composition according to one or more embodiments described in this specification in the manufacture of a medicament is also disclosed.

Use of the pharmaceutical composition according to one or more embodiments described in this specification in the manufacture of a medicament for the treatment of cancer and/or for boron neutron capture therapy is also disclosed.

A method for preparing the pharmaceutical composition according to one or more embodiments described in this specification is disclosed. The method may comprise
mixing BPA with a 2HA to form an aqueous solution of the BPA and the 2HA,
optionally mixing a polyol with the BPA and the 2HA or adding a polyol to the aqueous solution, and
adjusting the pH of the aqueous solution to a pH in the range of 6.5 to 8.5.

In an embodiment, the method may comprise mixing BPA with a 2HA to form an aqueous solution of the BPA, the 2HA,
optionally mixing a polyol with the BPA and the 2HA or adding a polyol to the aqueous solution,
adjusting the pH of the aqueous solution with an alkali to completely dissolve the BPA, and
adjusting the pH of the aqueous solution to a pH in the range of 6.5 to 8.5.

The pH of the aqueous solution may be adjusted to a pH in the range of 6.5 to 8.5 with an acid.

BPA may be mixed with an aqueous solution of the 2HA to form the aqueous solution of the BPA and the 2HA. Alternatively, it may be possible to mix BPA, the 2HA and water to form the aqueous solution of the BPA and the 2HA.

BPA may be mixed with an aqueous solution of the 2HA and the polyol to form the aqueous solution of the BPA, the 2HA and the polyol. Alternatively, it may be possible to mix BPA, the 2HA, the polyol and water to form the aqueous solution of the BPA, the 2HA, and the polyol. Alternatively, BPA may be mixed with an aqueous solution of the 2HA to form the aqueous solution of the BPA and the 2HA, and add the polyol to the aqueous solution.

The pharmaceutical composition prepared by the method may thus be an aqueous solution.

The aqueous solution may be prepared e.g. by mixing BPA and the 2HA in water, adding an acid to the obtained alkaline aqueous solution to adjust the pH of the obtained alkaline aqueous solution, for example to a substantially neutral pH or to a substantially physiological pH. In an embodiment, the aqueous solution may be prepared e.g. by mixing BPA, the 2HA and optionally the polyol in water, adding an acid to the obtained alkaline aqueous solution to adjust the pH of the obtained alkaline aqueous solution, for example to a substantially neutral pH or to a substantially physiological pH. In an embodiment, an alkali may be added before the pH adjustment to completely dissolve the BPA. The temperature at the time of the production of the aqueous solution is not particularly limited, and it can be simply performed at room temperature. Heating (for example, to 60° C.) as in the production of the BPA fructose complex solution is not required. The aqueous solution may further be filter sterilized, if necessary or desired.

The pH of the aqueous solution may be adjusted e.g. to a higher pH, for example to a pH in the range of 7.5-8, with an alkali to completely dissolve the BPA. The need to adjust the pH of the aqueous solution to completely dissolve the BPA may depend on the exact composition of the aqueous solution.

The pH of the aqueous solution may be adjusted to a pH in the range of 6.5 to 8.5 using a suitable agent, such as an acid.

The acid to be added for adjusting the pH of the aqueous solution after mixing BPA with water and 2HA is not particularly limited as long as it is an acid which is suitable for the purpose and can be used for medical purposes, i.e. is pharmaceutically acceptable. The acid may be e.g. hydrochloric acid.

The alkali to be added for completely dissolving the BPA is not particularly limited as long as it is an alkali which is suitable for the purpose and can be used for medical purposes, i.e. is pharmaceutically acceptable. The alkali may be e.g. sodium hydroxide, potassium hydroxide, and/or sodium carbonate.

The molar ratio of 2HA:BPA may be in the range of 0.5-3, about 0.5, in the range of 0.5-1, about 1, in the range of 1-1.5, in the range of 1-2, about 2, in the range of 2-3, or about 3.

In an embodiment, the molar ratio of 2HA:BPA is 1.

In the context of the method, the molar ratios and/or amounts of the 2HA, of the BPA, and optionally of the polyol may be any molar ratio and/or amount described in the context of the pharmaceutical composition.

In an embodiment, the molar proportions and/or molar amounts of the BPA, the 2HA, and the polyol are such that the sum of the molar amounts of BPA and polyol is about equal to or larger than the molar amount of BPA.

In the embodiments herein, [2HA] is the molar amount of the 2HA, [polyol] is the molar amount of the polyol, [BPA] is the molar amount of the BPA, and x is the multiplication sign. In other words, 3×[BPA] may be understood as referring to three times the molar amount of the BPA. If the polyol comprises or is a mixture of two or more different polyols, then [polyol] is the sum of the molar amounts of the two or more different polyols.

In an embodiment, [2HA]+[polyol]>1.5×[BPA]. In an embodiment, [2HA]+[polyol]>2×[BPA]. In an embodiment, [BPA]<[2HA]+[polyol]<3×[BPA]. In an embodiment, [BPA]<[2HA]+[polyol]<2.5×[BPA]. In an embodiment, [BPA]<[2HA]+[polyol]<2×[BPA].

In an embodiment, [2HA]+[polyol]≥0.8×[BPA]. In an embodiment, [2HA]+[polyol]≥0.9×[BPA]. In an embodiment, [2HA]+[polyol]≥1.0×[BPA]. In an embodiment, [2HA]+[polyol]≥1.5×[BPA]. In an embodiment, [2HA]+[polyol]≥2.0×[BPA].

In an embodiment, 0.8×[BPA]≤[2HA]+[polyol]≤5.0×[BPA]. In an embodiment, 0.9×[BPA]≤[2HA]+[polyol]≤4.0×[BPA]. In an embodiment, 1.0×[BPA]≤[2HA]+[polyol]≤3.0×[BPA]. In an embodiment, 1.5×[BPA]≤[2HA]+[polyol]≤2.5×[BPA]. In an embodiment, 2.0×[BPA]≤[2HA]+[polyol]≤2.2×[BPA].

In an embodiment, [2HA]+[polyol]=2.1×[BPA].

In an embodiment, no polyethylene glycol, polyol, saccharide, fructose, mannitol or sorbitol is added to the aqueous solution and/or to the pharmaceutical composition. In other words, the pharmaceutical composition is free of polyethylene glycol, polyol, saccharide, fructose, mannitol and/or sorbitol.

In an embodiment, a polyol is added to the aqueous solution and/or to the pharmaceutical composition.

In an embodiment, the polyol is mannitol.

In an embodiment, the molar ratio of polyol:BPA may be in the range of 0.5-3, about 0.5, in the range of 0.5-1, about 1, about 1.1, in the range of 1-1.5, in the range of 1-2, about 2, in the range of 2-3, or about 3.

In an embodiment, the molar ratio of polyol:BPA is 11:10.

In an embodiment, the molar ratios of BPA:2HA:polyol are 10:10:11.

The molar ratio of Tris(hydroxymethyl)aminomethane (Tris):L-p-boronophenylalanine may be in the range of 0.5-3, about 0.5, in the range of 0.5-1, about 1, in the range of 1-1.5, in the range of 1-2, about 2, in the range of 2-3, or about 3.

In the embodiments below, [Tris] is the molar amount of Tris, [mannitol] is the molar amount of mannitol, and [BPA] is the molar amount of L-p-boronophenylalanine.

In an embodiment, [Tris]+[mannitol]>[L-p-boronophenylalanine], wherein [Tris] is the molar amount of Tris, [mannitol] is the molar amount of mannitol, and [L-p-boronophenylalanine] is the molar amount of L-p-boronophenylalanine. In an embodiment, [Tris]+[mannitol]>1.5×[L-p-boronophenylalanine]. In an embodiment, [Tris]+[mannitol]>2×[L-p-boronophenylalanine]. In an embodiment, [Tris]+[mannitol]<3×[L-p-boronophenylalanine]. In an embodiment, [Tris]+[mannitol]<2.5×[L-p-boronophenylalanine]. In an embodiment, [Tris]+[mannitol]<2×[L-p-boronophenylalanine].

In an embodiment, the pharmaceutical composition comprises L-p-boronophenylalanine, Tris and mannitol in molar proportions 10:10:11.

In an embodiment, the pharmaceutical composition comprises L-p-boronophenylalanine, Tris and mannitol in molar proportions 10:10:11, and the pH of the composition is between pH 6.5-7.5, or about pH 7.4 or a physiological pH or a substantially physiological pH. In an embodiment, [Tris]+[mannitol]≥0.8×[BPA]. In an embodiment, [Tris]+[mannitol]≥0.9×[BPA]. In an embodiment, [Tris]+[mannitol]≥1.0×[BPA]. In an embodiment, [Tris]+[mannitol]≥1.5×[BPA]. In an embodiment, [Tris]+[mannitol]≥2.0×[BPA].

In an embodiment, 0.8×[BPA]≤[Tris]+[mannitol]≤5.0×[BPA]. In an embodiment, 0.9×[BPA]≤[Tris]+[mannitol]≤4.0×[BPA]. In an embodiment, 1.0×[BPA]≤[Tris]+[mannitol]≤3.0×[BPA]. In an embodiment, 1.5×[BPA]≤[Tris]+[mannitol]≤2.5×[BPA]. In an embodiment, 2.0×[BPA]≤[Tris]+[mannitol]≤2.2×[BPA].

In an embodiment, [Tris]+[mannitol]=2.1×[BPA].

In the above embodiments indicating the molar ratios and/or molar amounts, the pharmaceutical composition may be in the form of an aqueous solution or a dry formulation.

In an embodiment, the method may comprise
mixing BPA with the 2HA to form an aqueous solution of the BPA and the 2HA,
optionally adding the polyol to the aqueous solution, and
adjusting the pH of the aqueous solution to a pH in the range of 6.5 to 8.5.

In an embodiment, the method comprises
mixing L-p-boronophenylalanine and Tris(hydroxymethyl)aminomethane (Tris), to form an aqueous solution,
mixing mannitol with the BPA and the 2HA or adding mannitol to the aqueous solution,
optionally adding an agent for adjusting the pH, such as an alkali, to the aqueous solution to completely dissolve the BPA, and
adjusting the pH of the aqueous solution with an acid to a pH of about pH 7.4 or a physiological pH or a substantially physiological pH.

In an embodiment, the alkali is sodium hydroxide, and/or the acid is hydrochloric acid.

In an embodiment, in the method the concentration of the BPA in the aqueous solution is in the range of 30-120 g/L and the pH of the aqueous solution is about pH 7.4 or a physiological pH or a substantially physiological pH.

The method may further comprise drying the aqueous solution, thereby obtaining a dry formulation of the pharmaceutical composition.

A pharmaceutical composition obtainable by one or more embodiments of the method is also disclosed.

A method for producing the pharmaceutical composition as an aqueous solution according to one or more embodiments described in this specification is further disclosed. The method may comprise
providing the pharmaceutical composition as a dry formulation, such as a dry formulation according to one or more embodiments described in this specification,
mixing the pharmaceutical composition as the dry formulation with water and optionally with one or more compounds selected from hydrochloric acid, sodium chloride, acetic acid, sodium acetate, polyethylene glycol, a polyol, a saccharide, fructose, mannitol and sorbitol, thereby obtaining the pharmaceutical composition as an aqueous solution.

The concentration of the BPA in the aqueous solution obtainable by the method for producing the pharmaceutical composition as an aqueous solution may in the range of 30-120 g/L. The pH of the aqueous solution may be about pH 7.4, or a physiological pH or a substantially physiological pH.

In this aspect, the composition may subsequently be ready for use in therapy. The dry formulation may thus be reconstituted by mixing it with water and optionally with one or more compounds.

The method according to this aspect may be performed e.g. prior to administration to a subject.

In an embodiment, no polyethylene glycol, polyol, saccharide, fructose, mannitol or sorbitol is added to the aqueous solution and/or to the pharmaceutical composition.

In an embodiment, the pharmaceutical composition comprises a therapeutically effective amount of BPA.

The term “therapeutically effective amount” or “effective amount” of the conjugate may be understood as referring to the dosage regimen for achieving a therapeutic effect. The therapeutically effective amount may be selected in accordance with a variety of factors, including the age, weight, sex, diet and medical condition of the patient, the severity of the disease, and pharmacological considerations, such as the activity, efficacy, pharmacokinetic and toxicology profiles of the particular conjugate used. The therapeutically effective amount can also be determined by reference to standard medical texts, such as the Physicians Desk Reference 2004. The patient may be male or female, and may be an infant, child or adult.

The term “treatment” or “treat” is used in the conventional sense and means attending to, caring for and nursing a patient with the aim of combating, reducing, attenuating or alleviating an illness or health abnormality and improving the living conditions impaired by this illness, such as, for example, with a cancer disease.

The pharmaceutical composition may comprise or be a composition for e.g. intravenal, intraarterial, and/or intra-tumoral (i.t.) administration or for direct injection into tissue. Administration of the pharmaceutical composition may be effected in different ways, e.g. by intravenous, intraperitoneal, intraarterial, or intra-tumoral administration.

Use of a 2-hydroxy amine compound (2HA) or a pharmaceutically acceptable salt thereof according to one or more embodiments described in this specification in dissolving BPA, thereby forming a pharmaceutical composition comprising BPA as an aqueous solution, is also disclosed.

The 2HA or the pharmaceutically acceptable salt thereof may be used in dissolving BPA in the absence of polyethylene glycol, a polyol, saccharide, fructose, mannitol and/or sorbitol.

In an embodiment, the 2HA or the pharmaceutically acceptable salt thereof may be used in dissolving BPA in the presence of polyethylene glycol, a polyol, saccharide, fructose, mannitol and/or sorbitol.

In an embodiment, the 2HA or the pharmaceutically acceptable salt thereof may be used in dissolving BPA in the presence of mannitol.

In the context of the use, the pharmaceutical composition may be the pharmaceutical composition according to one or more embodiments described in this specification. In the context of the use, the BPA, the 2HA, the polyol, and any molar amounts and/or ratios may be as described above in the context of the pharmaceutical composition and of the method(s).

### EXAMPLES

Although all BPA used in Examples 1-5 were synthesized by a conventional method using boron in which the proportion of ¹⁰B to the whole boron is about 20%, the isotope of boron does not affect the results obtained in the Examples and they are also applicable to BPA containing any ratio of ¹⁰B.

### Example 1. Solubilization of BPA

BPA (L-p-boronophenylalanine, Katchem, Praha, Czech Republic) was weighed to a set of test tubes, 15 mg each (72 μmol). At room temperature (RT), 0.5 ml each of various aqueous solutions as listed below, were added into parallel test tubes and mixed vigorously. After 2 hours, the results of visual inspection of BPA dissolution and pH were as follows:

### Water

deionized water—no dissolution

### Strong Base

0.5 M NaOH—complete dissolution

### Organic Alcohols

96% ethanol (v/v)—no dissolution
0.5 M propylene glycol—no dissolution

### Neutral Solutions

0.12 M L-histidine (neutral pH)—no dissolution
0.5 M L-serine (neutral pH)—no dissolution
0.5 M sodium-potassium tartrate (neutral pH)—partial dissolution

### Basic Compounds

0.5 M L-arginine (pH over 11)—partial dissolution

### Strong Base Followed by Hydroxy Amino Acid

0.25 ml 0.5 M NaOH followed by 0.5 ml 0.5 M L-serine—complete dissolution

### 2-Hydroxy Amine Compound (2HA)

0.5 M Tris(Trizma base, pH over 10)—complete dissolution
0.5 M Tris-HCl (pH 8)—partial dissolution

### Example 2. Aqueous BPA Formulations at Physiological pH

BPA-Tris-HCl pH 8: 15 mg BPA was dissolved in 0.45 ml of 0.5 M Trizma base at RT, with complete dissolution of BPA. 20 μl of 5 M HCl was added, after which pH was about 8 and the BPA was completely dissolved.

BPA-Tris-HCl pH 7.5: 15 mg BPA was dissolved in 0.45 ml of 0.5 M Trizma base at RT, with complete dissolution of BPA. 20 μl of 5 M HCl and 20 μl of 1 M HCl were added, after which pH was about 7.5 and the BPA was completely dissolved.

### Example 3. BPA Formulations with Fructose

BPA-Tris-HCl-fructose pH 8: To the BPA-Tris-HCl pH 8 formulation above, 0.5 ml 0.5 M fructose was added and the BPA was completely dissolved.

BPA-Tris-HCl-fructose pH 7.5: To the BPA-Tris-HCl pH 7.5 formulation above, 0.5 ml 0.5 M fructose was added and the BPA was completely dissolved.

### Example 4. Freeze-Dried BPA Formulations

BPA-Tris-HCl pH 8 freeze-dried: The BPA-Tris-HCl pH 8 formulation was freeze-dried. After adding 355 μl of MQ to the lyophilizate, the BPA was completely dissolved.

BPA-Tris-HCl pH 7.5 freeze-dried: The BPA-Tris-HCl pH 7.5 formulation was freeze-dried. After adding 355 μl of MQ to the lyophilizate, the BPA was completely dissolved.

### Example 5. Freeze-Dried BPA Formulations with Fructose

BPA-Tris-HCl pH 8 freeze-dried+fructose: The BPA-Tris-HCl pH 8 formulation was freeze-dried. After adding 355 μl of MQ and 145 μl 0.5 M fructose to the lyophilizate, the BPA was completely dissolved.

BPA-Tris-HCl pH 7.5 freeze-dried+fructose: The BPA-Tris-HCl pH 7.5 formulation was freeze-dried. After adding 355 μl of MQ and 145 μl 0.5 M fructose to the lyophilizate, the BPA was completely dissolved.

All BPA used in Examples 6 onwards was enriched with ¹⁰B (¹⁰B-L-p-boronophenylalanine, hereinafter interchangeably either BPA or ¹⁰BPA) obtained from Interpharma Praha, Prague, Czech Republic. All other reagents were from Sigma unless stated otherwise. RT=room temperature at about 20-23° C.

### Example 6. Comparison of BPA-Tris-Mannitol and BPA-Tris-Fructose Formulations at Physiological and Slightly Alkaline pH

30 mg aliquots of ¹⁰BPA were dissolved in 0.5 M aqueous Tris base (Sigma) at +37° C., with Tris:BPA molar ratio of 2:1, and after complete dissolution pH was adjusted with dilute HCl to either pH 7.4 or pH 8.0. The aliquots were then lyophilized. To compare potential of mannitol and fructose to keep lyophilized BPA-Tris in solution without precipitation, the aliquots were dissolved in aqueous sugar solution (either mannitol or fructose with sugar:BPA molar ratio of 11:10) to 30 g BPA/L and incubated in RT. After 1 day, the formulation with fructose at pH 7.4 started to precipitate. After 4 days, also the formulation with fructose at pH 8.0 started to precipitate, while the formulations with mannitol both at pH 7.4 and pH 8.0 were clear solutions without precipitation.

30 mg aliquots of ¹⁰BPA, together with either fructose or mannitol (sugar:BPA molar ratio of 11:10), were dissolved in 0.9 ml of 0.5 M aqueous Tris base (Sigma) at +37° C. (Tris:BPA molar ratio of 3:1), and after complete dissolution pH was adjusted with dilute HCl to pH 7.4. The aliquots were then lyophilized. To compare potential of mannitol and fructose to keep the lyophilized BPA-Tris-sugar in solution without precipitation, the aliquots were dissolved in water to 30 g BPA/L and incubated in RT. After 1 day, the formulation with fructose at pH 7.4 had started to precipitate. In contrast, after 15 days, the formulation with mannitol was still a clear solution without precipitation.

Thus, mannitol was superior to fructose in keeping BPA-Tris formulations in aqueous solution at pH 7.4-8.0 for a prolonged time without precipitation.

### Example 7. BPA Formulations with Bis-Tris and Triethanolamine

30 mg aliquots of ¹⁰BPA were combined with 0.9 ml each of either 0.5 M aqueous Bis-Tris base (Sigma) or 0.5 M aqueous triethanolamine (Sigma). The molar ratios of both Bis-Tris:BPA and triethanolamine:BPA were thus 3:1. The aliquots were adjusted with 3 M NaOH to pH>8, after which they were incubated for 15-30 min at +37° C. to completely dissolve the BPA. Then the pH was adjusted with dilute HCl to either pH 7.4 or pH 8.0 in different aliquots and the aliquots were then lyophilized. The aliquots were dissolved in in water to 30 g BPA/L and incubated in RT. After 2 h, the formulation with triethanolamine at pH 7.4 started to precipitate. In contrast, after 15 days, all the other formulations (BPA-Bis-Tris pH 7.4, BPA-Bis-Tris pH 7.4, and BPA-Bis-Tris pH 7.4) were still clear solutions without precipitation.

30 mg aliquots of ¹⁰BPA were combined with different amounts of aqueous Bis-Tris base (Sigma) to molar ratios of Bis-Tris:BPA of 3:1, 2:1 and 1:1, with or without mannitol at molar ratio 11:10 of mannitol:BPA. The aliquots were adjusted with 3 M NaOH to pH>8, after which they were incubated at +37° C. to completely dissolve. Then the pH was adjusted with dilute HCl to pH 7.4 and the volume was adjusted with water to 30 g BPA/L, after which the solutions were incubated in RT. After 7 days, the formulation with 1:1 molar ratio of Bis-Tris:BPA at pH 7.4 started to precipitate. In contrast, all the other formulations (BPA-Bis-Tris pH 7.4, BPA-Bis-Tris pH 7.4, and BPA-Bis-Tris pH 7.4) were still clear solutions without precipitation.

In conclusion, BPA could be formulated as an aqueous solution with various 2HA's, with Bis-Tris at pH 7.4-8.0, and with triethanolamine at pH 8.0, for a prolonged time without precipitation. Molar ratios of Bis-Tris:BPA between 1:1-3:1 could dissolve BPA at pH 7.4, while adding mannitol to the formulation with the molar ratio of 1:1 helped to keep the BPA at pH 7.4 dissolved for a prolonged time without precipitation.

### Example 8. BPA-Tris-Mannitol Formulations at Different pH

Aliquots of BPA-Tris-mannitol were prepared, each by combining 288 μmol ¹⁰BPA, 288 μmol Tris base, 318 μmol mannitol (BPA:Tris:mannitol molar ratios 10:10:11) and 600 μmol NaOH in water at +37° C. to completely dissolve. The pH was adjusted with dilute HCl to either pH 7.2, 7.5 or 8.0, and volume adjusted with water to 30 g BPA/L. Parallel aliquots at each pH were either directly incubated in RT, or lyophilized, redissolved with water to 30 g BPA/L, and then incubated in RT. The formulation at pH 7.2 started to precipitate both before and after lyophilization and redissolving, while the other formulations at pH 7.5 and pH 8.0 remained clear solutions without precipitation.

In contrast, BPA-mannitol formulations prepared without Tris base but otherwise similarly as above, precipitated after adjusting pH to either 7.3, 7.4, 7.5, 7.6, 7.7 or 7.8. These aliquots were prepared by combining 288 μmol ¹⁰BPA, 318 μmol mannitol (BPA: mannitol molar ratios 10:11) and 600 μmol NaOH in water at +37° C. to completely dissolve, followed by pH adjustment with dilute HCl.

In conclusion, BPA-Tris-mannitol formulations could be readily adjusted to pH values over pH 7.2, while otherwise similar BPA-mannitol formulations without Tris could not be adjusted to pH values between pH 7.3-7.8.

### Example 9. High-Concentration BPA Formulations with Tris

The same procedure as above was used to prepare BPA-Tris-mannitol lyophilisates and solutions at 60 g BPA/L and 90 g BPA/L concentrations at pH 7.4, with BPA:Tris:mannitol molar ratios of 10:10:11. A high concentration BPA-Tris-mannitol lyophilisate and solution at 120 g BPA/L concentration at pH 7.4 was further prepared as described below in Example 11. All these formulations were fully soluble in both +4° C. and RT without precipitation. In contrast, BPA-sorbitol and BPA-fructose formulations without Tris could not be prepared at 120 g BPA/L as described below.

An attempt to prepare high-concentration BPA-sorbitol was made by combining 240 mg (1153 μmol) ¹⁰BPA and 252 mg sorbitol (1383 μmol; BPA:sorbitol molar ratio 10:12) in 2 mL volume to achieve 120 g BPA/L, and 600 μmol NaOH in water at +37° C. However, the BPA-sorbitol could not be dissolved at this concentration and it remained as a precipitate.

An attempt to prepare high-concentration BPA-fructose was made by combining 240 mg (1153 μmol) ¹⁰BPA and 229 mg fructose (1268 μmol; BPA:fructose molar ratio 10:11) in nearly 2 mL volume to achieve 120 g BPA/L, and aqueous NaOH was with mixing at +37° C. until pH was above 10. However, the BPA-fructose could not be dissolved at this concentration and it remained as a precipitate.

### Example 10. BPA-Tris-Sorbitol Formulation

BPA-Tris-sorbitol was prepared by combining 60 mg (288 μmol) ¹⁰BPA, 288 μmol Tris base, 317 μmol sorbitol (BPA:Tris:sorbitol molar ratios 10:10:11) and 225 μmol NaOH in water at +37° C. to completely dissolve. The pH was adjusted with dilute HCl to pH 7.4 and volume adjusted with water to 30 g BPA/L (2 ml). The formulation remained as a clear solution at both +4° C. and RT without precipitation.

### Example 11. BPA Formulations with Fructose, Mannitol, Tris-Mannitol, or Sorbitol, at pH 7.4-8

GMP-grade ¹⁰BPA was from Interpharma Praha, Prague, Czech Republic. Other essential reagents were as follows:

| Reagent | Producer | Product | Cat. No. |
|---|---|---|---|
| Tris base | Sigma- | Trizma ® base BioPerformance | T6066 |
| | Aldrich | Certified, meets EP, USP testing | |
| | | specifications, ≥99.9% | |
| Fructose | Sigma- | D-Fructose tested according to | 47748 |
| | Aldrich | Ph. Eur. | |
| Mannitol | Sigma- | D-Mannitol, meets EP, FCC, | M8429 |
| | Aldrich | USP testing specifications | |
| Sorbitol | Sigma- | D-Sorbitol BioReagent, suitable | S3889 |
| | Aldrich | for cell culture | |
| NaOH | Merck | Sodium hydroxide pellets | 1.06482 |
| | | EMPROVE ® ESSENTIAL Ph. | |
| | | Eur., BP, FCC, JP, NF, E 524 | |
| HCl | Merck | Hydrochloric acid 37%, | 1.00314 |
| | | EMPROVE ® ESSENTIAL Ph. | |
| | | Eur., BP, JP, NF | |
| Water | Cytiva | HyPure Cell Culture Grade | SH30529 |
| | | Water, endotoxin-free | |

Standard BPA-fructose formulation is called BPA-F. Formulation with BPA-Tris-mannitol, molar proportions 1:1:1.1, is called BPA-T. Compound 2 is BPA-T (30 g BPA/L) pH 7.4 and Compound 5 is BPA-T (120 g BPA/L) pH 7.6.

Compound 1 (BPA-F pH 7.4) was prepared at 30 g BPA/L essentially according to van Rij et al. 2005 (Am. J. Health-Syst. Pharm. 62:2608-10). Two lots were prepared to provide fresh BPA-F for two experiments.

Lot 1: 149.9 mg BPA was added into 2.5 mL of 0.54 M NaOH. 30 μL of 2 M NaOH was added and mixed until completely dissolved and clear, after which 142.8 mg of fructose was added and mixed. At this point, the pH was 10.45. 320 μL 2 M HCl was added to bring the pH to 7.4. 2.15 mL of endotoxin-free water was added to bring the volume to 5 mL, and the product was sterile filtered.

Lot 2: 90.4 mg BPA was added into 1.5 mL of 0.54 M NaOH. 20 μL of 2 M NaOH was added and mixed until completely dissolved and clear, after which 85.2 mg of fructose was added and mixed. At this point, the pH was 10.49. 192 μL 2 M HCl was added to bring the pH to 7.43. 1.288 mL of endotoxin-free water was added to bring the volume to 3 mL, and the product was sterile filtered.

Compound 2 (BPA-T pH 7.4) was prepared at 30 g BPA/L in two lots for two experiments.

Lot 1: 90 mg BPA and 86.5 mg mannitol were dissolved into solution of 2.022 mL endotoxin-free water with 865 μL 0.5 M Tris base and 113 μL 3 M NaOH, after which the pH was 8.71. 158 μL 2 M HCl was added to bring the pH to 7.42. The product was sterile filtered, freezed and lyophilized. Dissolution into 3 mL endotoxin-free water and sterile filtration yielded the ready product.

Lot 2: 149.8 mg BPA and 144.6 mg mannitol were dissolved into solution of 3.375 mL endotoxin-free water with 1.44 mL 0.5 M Tris base and 188 μL 3 M NaOH, after which the pH was 8.63. 265 μL 2 M HCl was added to bring the pH to 7.42. The product was sterile filtered, freezed and lyophilized. Dissolution into 5 mL endotoxin-free water and sterile filtration yielded the ready product.

Compound 3 (BPA-sorbitol pH 7.4) was prepared at 30 g BPA/L essentially according to JP2009051766. Into a solution of 2.4 mL endotoxin-free water, 450 μL 1 M NaOH and 15 μL 3 M NaOH, 90.4 mg BPA and 94.3 mg sorbitol were added and mixed. The mixture took 51 min at 37° C. to completely dissolve, after which the pH was 8.68. 8 μL 2 M HCl and 52 μL 1 M HCl were added to bring the pH to 7.43. 77 μL of endotoxin-free water was added to bring the volume to 3 mL, and the product was sterile filtered.

Compound 4 (BPA-mannitol pH 8) was prepared at 30 g BPA/L essentially according to Halbert et al. 2013 (Eur. J. Pharm. Sci. 48:735-9). 90.1 mg BPA and 99.2 mg mannitol were added into 2 mL endotoxin-free water and mixed. 185 μL 3 M NaOH was added and mixed at 37° C. until completely dissolved, after which the pH was 9.01. 50 μL 2 M HCl was added to bring the pH to 7.93. 765 μL of endotoxin-free water was added to bring the volume to 3 mL, and the product was sterile filtered, freezed and lyophilized. Dissolution into 3 mL endotoxin-free water and sterile filtration yielded the ready product.

Compound 5 (BPA-T pH 7.6) was prepared at 120 g BPA/L. 120 mg BPA and 115.3 mg mannitol were dissolved into solution of 700 μL endotoxin-free water with 1152 μL 0.5 M Tris base and 125 μL 3 M NaOH, after which the pH was 8.37. 148 μL 2 M HCl was added to bring the pH to 7.63. The product was sterile filtered, freezed and lyophilized. Dissolution into 1 mL endotoxin-free water and sterile filtration yielded the ready product.

The ready test substances were stored at room temperature until use. The final compositions of the substances were according to the Table below:

| | BPA | Sugar* | Na⁺ | Cl⁻ | Tris | | | |
|---|---|---|---|---|---|---|---|---|
| Test | g/L | g/L | g/L | g/L | g/L | | | |
| compound | (mol/L) | (mol/L) | (mol/L) | (mol/L) | (mol/L) | Sugar:BPA | Tris:BPA | pH |
| BPA-F | 30 | 29 | 6.5 | 4.5 | | 1.1 | | 7.4 |
| | (0.14) | (0.16) | (0.28) | (0.13) | | | | |
| BPA-T | 30 | 29 | 2.6 | 3.7 | 17 | 1.1 | 1.0 | 7.4 |
| | (0.14) | (0.16) | (0.11) | (0.11) | (0.14) | | | |
| BPA- | 30 | 31 | 3.8 | 0.8 | | 1.2 | | 7.4 |
| sorbitol | (0.14) | (0.17) | (0.17) | (0.02) | | | | |
| BPA- | 30 | 33 | 4.3 | 1.2 | | 1.3 | | 7.9 |
| mannitol | (0.14) | (0.18) | (0.19) | (0.03) | | | | |
| BPA-T | 120 | 115 | 8.6 | 10 | 70 | 1.1 | 1.0 | 7.6 |
| 120 g/L | (0.58) | (0.63) | (0.38) | (0.30) | (0.58) | | | |

*Sugar is fructose in BPA-F, mannitol in BPA-T, sorbitol in BPA-sorbitol and mannitol in BPA-mannitol.

The Table of BPA substance compositions reveals several advantages of BPA-T over the other BPA formulations.

BPA concentration and administered volume: BPA-T could be prepared both at the standard concentration of 30 g BPA/L and at a 4× higher concentration of 120 g BPA/L for successful i.v. administration to mice. The 4× higher concentration could be administered in 4× smaller volume in the i.v. injection. This results in lesser infusion volume stress for the recipient of the i.v. injection when using BPA-T with higher BPA concentration compared to the other BPA formulations.

Amount of sodium: BPA-T contained the smallest amount of sodium (Na⁺ ions) of all the BPA formulations, 2.2-2.6 g sodium for 30 g BPA, compared to 3.8-6.5 g for 30 g BPA in the BPA-F, BPA-sorbitol and BPA-mannitol formulations. This results in lesser sodium stress for the recipient of the i.v. injection when using BPA-T compared to the other BPA formulations.

Sugar:BPA ratio: BPA-T contained the smallest amount of sugar, 1.1 mol mannitol for 1 mol BPA, especially compared to 1.2-1.3 mol sugar for 1 mol BPA with BPA-sorbitol and BPA-mannitol formulations.

pH: BPA-T was formulated at the physiological pH of 7.4 with excellent solubility at 30-120 g BPA/L, whereas especially BPA-mannitol could not be formulated in soluble form below pH 7.9. This results in lesser pH stress for the recipient of the i.v. injection when using BPA-T compared to especially BPA-mannitol.

### Example 12. In Vivo Biodistribution of BPA Formulations in Tumor-Xenografted Mice

The in vivo study was initiated to test if BPA-T could be safely administered to mice in clinically relevant conditions and if the tumor localization and biodistribution of BPA-T in tumor-xenografted mice after intravenous (i.v.) dosing would be similar to BPA-F, BPA-mannitol and BPA-sorbitol.

HSC-2 head-and-neck cancer cells were obtained from the Japanese Cancer Research Resources Cell Bank (JCRB; https://cellbank.nibiohn.go.jp/english/). The cell line was established from a 69-year-old male patient with oral squamous cell carcinoma (Momose et al. 1989, J. Oral Pathol. Med. 18:391-5). The cells were cultured with standard procedures according to the cell bank's instructions. On the day of the cell delivery, the cells were collected for subcutaneous (s.c.) inoculation to mice in 100 μl of 50% Matrigel, 2 million cells/mouse.

The experiment was carried out at the TCDM (Turku Center for Disease Modeling), Turku, Finland, according to the ethical committee's approval. Tumor take rate was excellent (100%). Tumor growth was followed by palpation until average tumor size reached 200 mm³. The biodistribution experiment was thereafter performed on two dates.

1) Time course study with BPA-F and BPA-T. The first 24 tumor mice were divided into groups of three mice/group (n=3, average group tumor size 208 mm³, group average range 203-219 mm³, tumor size range 124-385 mm³). Half of the mice were given an i.v. bolus of BPA-F and half were given BPA-T (all in 200 μl volume with 30 g BPA/L). The average mouse weight in this experiment was 20.9 g (range 18.5-22.3 g) and the average dose was 287 mg BPA/kg (range 269-324 mg/kg). At time points of 1, 2, 4 and 8 hours, a group of three mice was sacrificed from both BPA-F and BPA-T cohorts and tissue samples were taken.

2) Biodistribution at two hours after administration with BPA-F, BPA-T (30 g/L and 120 g/L), BPA-mannitol, BPA-sorbitol and vehicle (PBS). The last 36 tumor mice were divided into groups of six mice/group (n=6, average group tumor size 203 mm³, group average range 196-218 mm³, tumor size range 100-414 mm³). Each group was given i.v. bolus of the test compound or vehicle in 200 μl volume, except for the 120 g/L BPA-T group, which was given the administration in 50 μl volume. The average mouse weight in this experiment was 21.4 g (range 18.4-24.9 g) and the average dose was thus 280 mg BPA/kg (range 241-326 mg/kg). At the time point of 2 hours, all groups were sacrificed and tissue samples were taken.

From all mice, the following tissue samples were prepared and weighed: tumor, blood, cheek muscle, skin, brain, liver and salivary glands. The samples were weighed and stored frozen until the analyses.

The in vivo study went according to the plan and no adverse events, abnormalities or signs of toxicity were observed with any of the test substances.

¹⁰B and ¹¹B localization in selected mouse tissue samples were analyzed at ALS Scandinavia, Luleå, Sweden. 212 samples were provided for the analyses. All tissue samples were analyzed from the 2-hour biodistribution study with BPA-F and BPA-T, while from all the other study groups only tumor, blood and cheek muscle samples were analyzed. All samples were analyzed successfully by the ICP-SFMS method (inductively coupled plasma sector field mass spectrometry; https://www.alsglobal.se/en/isotope-analysis/laboratory). Control samples from mice that had received vehicle (PBS) but not BPA, contained between 3-22 μg ¹⁰B/kg tissue (equal to 3-22 ppb) and 13-91 μg 11B/kg tissue. They had an average ¹⁰B:¹¹B ratio of 0.238, which gives ¹⁰B abundance of 19.2%, similar to the published natural ¹⁰B abundance (19.3%, Bentley & Hamer 1958, Nature 182:1156). In contrast, samples from mice that had received BPA contained between 1368-31211 μg ¹⁰B/kg tissue (equal to 1368-31211 ppb, or about 1-31 ppm) and 30-457 μg 11B/kg tissue, showing effective ¹⁰B accumulation. The results were thus as expected for highly ¹⁰B-enriched BPA administration.

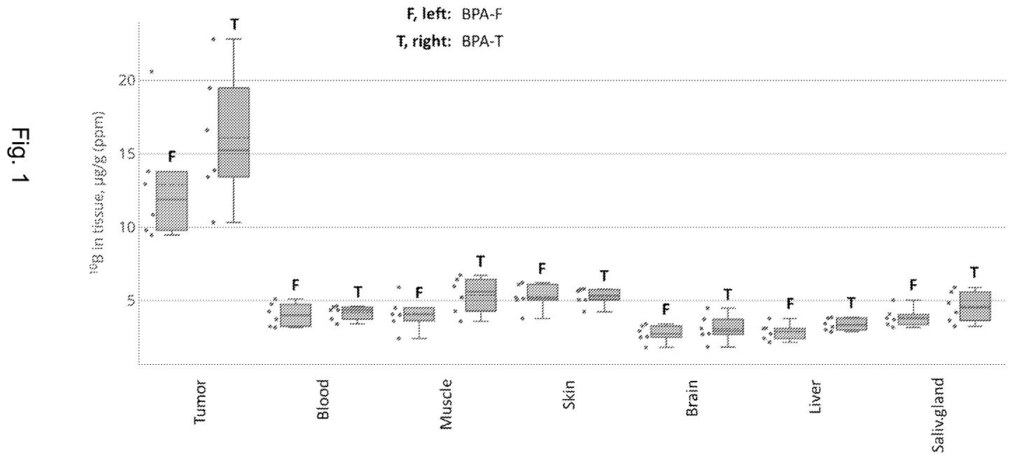

FIG. 1 shows comparison of BPA-T and BPA-F ¹⁰B concentrations in tumor mice two hours after dosing in all sampled tissues. Localization to tumor was the highest of all the tissues, between 9.5-20.6 μg/kg (on average 12.9 μg/kg) for BPA-F and between 10.3-22.8 μg/kg (on average 16.1 μg/kg) for BPA-T. Localization to brain was the lowest, on average 2.8 μg/kg for BPA-F and on average 3.1 μg/kg for BPA-T. The ¹⁰B concentration distributions were comparable between BPA-T and BPA-F both in the tumor and the tissues.

The achieved absolute ¹⁰B concentrations were clinically meaningful. In addition, although the dose of BPA (roughly 300 mg/kg) was an average value commonly utilized in animal trials, higher doses of up to 400-500 mg/kg are routinely used in clinical BNCT (Kankaanranta et al. 2011, Int. J. Radiat. Oncol. Biol. Phys. 80:369-76; Kankaanranta et al. 2011, Int. J. Radiat. Oncol. Biol. Phys. 82:e67-75).

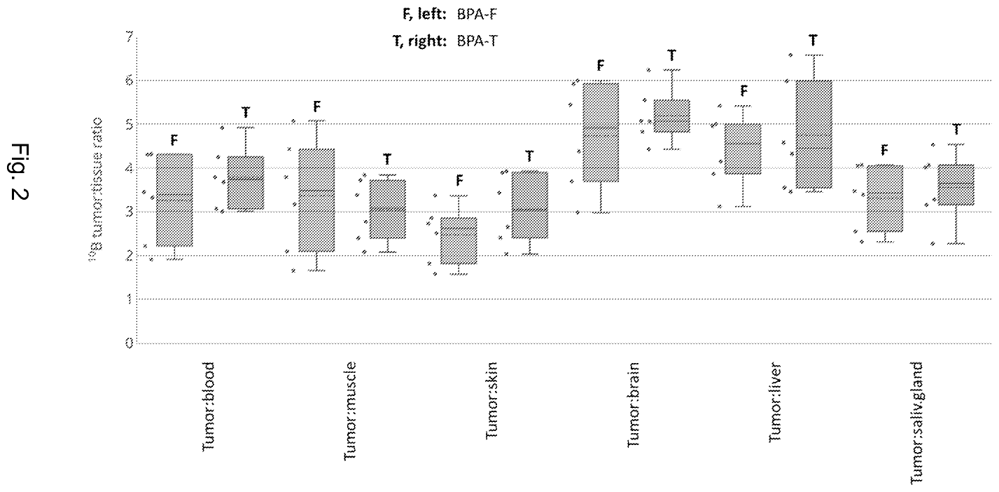

FIG. 2 shows comparison of BPA-T and BPA-F ¹⁰B tumor:tissue ratios in tumor mice two hours after dosing in all sampled tissues. No statistically significant differences in the ¹⁰B tumor:tissue ratios were detected between BPA-T and BPA-F with the t-test. Thus, the tissue biodistribution of BPA-T was comparable to BPA-F in the tumor-xenografted mice.

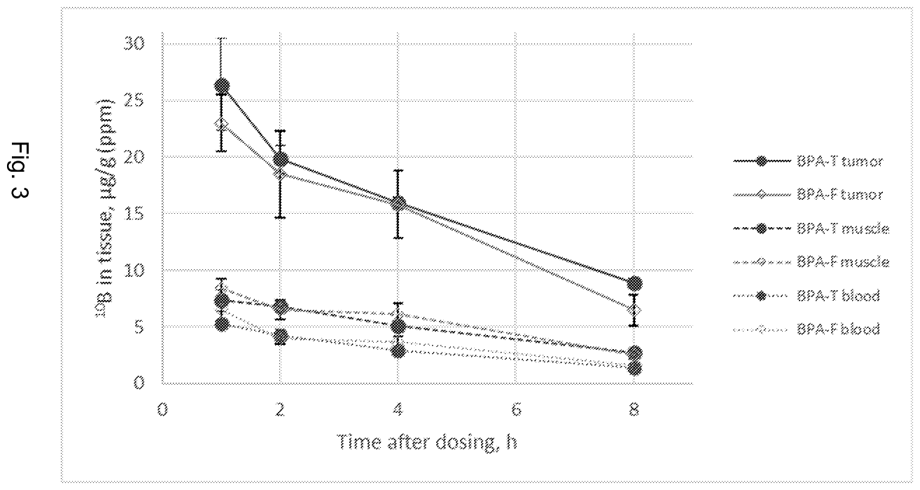

FIG. 3 shows the time series of BPA-T and BPA-F ¹⁰B concentrations between 1-8 hours after dosing. The graph shows that ¹⁰B concentrations decreased with similar speed during the study after both BPA-F and BPA-T administration. The tumor:blood ratio for BPA-T was on average 5.3 during the follow-up (range 4.7-6.2) and the tumor:muscle ratio was on average 3.2 (range 2.9-3.6).

Taken together, no significant differences could be detected between either spatial or temporal in vivo biodistribution of BPA-F and BPA-T in tumor-xenografted mice.

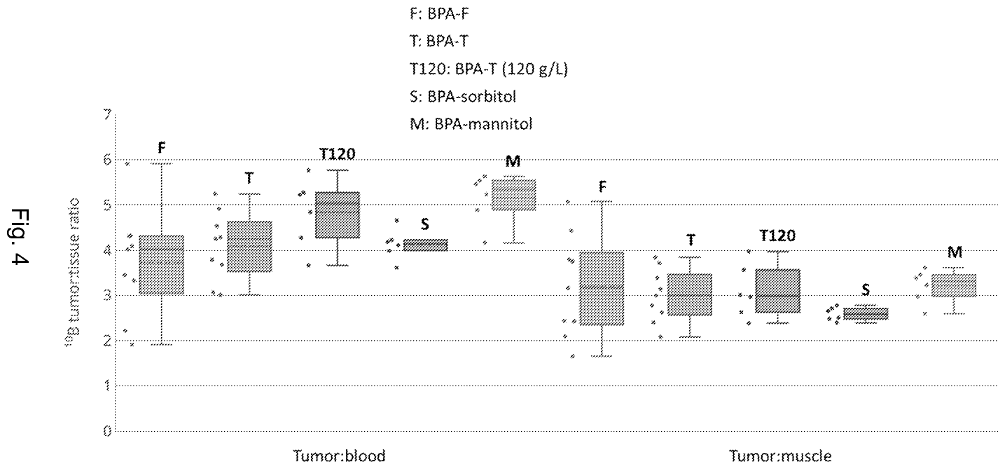

FIG. 4 shows comparison of the tumor:blood and tumor:muscle ¹⁰B ratios for BPA-T, BPA-F, BPA-mannitol and BPA-sorbitol with 30 g BPA/L formulations as well as for the BPA-T formulation with 120 g BPA/L. There were no significant differences between BPA-T and the other BPA formulations as evaluated by the Kruskal-Wallis test.

In conclusion, in the present study BPA-T showed similar biodistribution, tumor localization and rate of elimination from tissues when compared to BPA-F, BPA-mannitol and BPA-sorbitol.

### Example 13. Stability of BPA Formulations with Tris-mannitol

BPA-Tris-mannitol formulation aliquots were prepared with BPA:Tris:mannitol molar ratios of 10:10:11 at pH 7.4 as described above, lyophilized and stored at −20° C. or +4° C., or stored as liquid solution at +4° C. or +37° C. (elevated temperature for degradation studies). The lyophilized aliquots were dissolved with water to obtain solution concentration of 30 g BPA/L for the analyses at various time points.

RP-HPLC analysis was performed with a Gemini C18 column using a gradient from 100% A (0.1% trifluoracetic acid in water) to 100% B (acetonitrile) as follows: 100% A 5 min, 0-13% B in 19.5 min, 13-40% B in 10 min and 40-100% B in 5 min. Samples corresponding to 50 nmol amino acid were injected in A.

Standard compounds were analysed to establish elution times for both BPA and the potential degradation products L-phenylalanine and L-tyrosine. BPA eluted between 17.5-18.0 ml, L-phenylalanine eluted between 21.0-22.0 ml and L-tyrosine eluted between 16.5-17.0 ml. Thus, the analysis method could separate these components for their relative quantitation. Tyrosine was not observed in the BPA-Tris-mannitol formulations, while increasing amounts of phenylalanine were detected after prolonged incubation at elevated temperature of +37° C. The identity of BPA and phenylalanine were further confirmed by 1H-NMR spectroscopy against standard compounds. Therefore, it was established that the degradation of the BPA-Tris-mannitol formulation occurred to phenylalanine but not to tyrosine. The quantification of phenylalanine was adopted as the stability measure for the stability studies. BPA was quantitated based on absorbance at 256 nm and phenylalanine at 214 nm against standards. The BPA standard eluted at the same position as BPA in BPA-Tris-mannitol, BPA-mannitol, BPA-fructose and BPA-sorbitol formulations.

After a month's storage, the lyophilized formulations stored at −20° C. and +4° C. were both 100% BPA and did not contain any detectable amount of phenylalanine, and the liquid solution stored at +4° C. was 99.7% BPA and contained 0.3% phenylalanine (mol %). Thus, both lyophilized and solution BPA-Tris-mannitol formulations showed excellent stability and the lyophilized formulations were completely stable in these temperatures.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A product, a method, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A pharmaceutical composition comprising boronophenylalanine (BPA) or a pharmaceutically acceptable salt thereof and a 2-hydroxy amine compound (2HA) or a pharmaceutically acceptable salt thereof, wherein the 2HA is selected from the compounds set forth in any one of the formulas I to II or a pharmaceutically acceptable salt thereof, or any combination or mixture thereof: 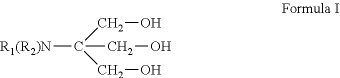
wherein R₁ and R₂ are each independently either H or selected from hydroxy-C₁-C₆-alkyl, 2-hydroxyethyl, 2,3,4,5,6-pentahydroxyhexyl, carboxy-C₁-C₆-alkyl, acetyl, C₁-C₆-alkyl, sulfo-C₁-C₆-alkyl, 3-sulfopropyl, 2-hydroxy-3-sulfopropyl, 1-sulfo-2-propanyl, 2-sulfoethyl, 3-[2-hydroxy-1,1-bis(hydroxymethyl)ethylamino]-C₁-C₆-alkyl, and 3-[2-hydroxy-1,1-bis(hydroxymethyl)ethylamino]propyl; 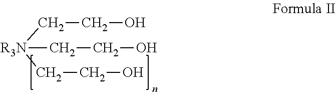
wherein n is 0 or 1;
provided that when n is 1, then R₃ is absent, and when n is 0, then R₃ is selected from H, hydroxy-C₁-C₆-alkyl, 2-hydroxyethyl, carboxy-C₁-C₆-alkyl, acetyl, C₁-C₆-alkyl, sulfo-C₁-C₆-alkyl and 2-sulfoethyl.

2. The pharmaceutical composition according to claim 1, wherein the BPA is L-p-boronophenylalanine or a pharmaceutically acceptable salt thereof.

3. The pharmaceutical composition according to claim 1, wherein the 2HA is selected from the following compounds, pharmaceutically acceptable salts thereof, and any mixtures and combinations thereof:
Tris(hydroxymethyl)aminomethane (Tris),
2-[Bis(2-hydroxyethyl)amino]-2-(hydroxymethyl)propane-1,3-diol (Bis-Tris),
(2-hydroxyethyl)amino-tris(hydroxymethyl)methane,
N-(Tris(hydroxymethyl)methyl)glycine (Tricine),
2-(Dimethylamino)-2-(hydroxymethyl)propane-1,3-diol (N,N-dimethyl-Tris),
1-Deoxy-1-([1,3-dihydroxy-2-(hydroxymethyl)-2-propanyl]amino)hexitol,
2-{[1,3-Dihydroxy-2-(hydroxymethyl)-2-propanyl]amino}-1-propanesulfonic acid,
[Tris(hydroxymethyl)methylamino]propanesulfonic acid (TAPS),
3-[N-Tris(hydroxymethyl)methylamino]-2-hydroxypropanesulfonic acid (TAPSO),
2-{[1,3-Dihydroxy-2-(hydroxymethyl)propan-2-yl]amino}ethane-1-sulfonic acid (TES),
Bis-Tris propane (BTP),
Diethanolamine (DEA),
Triethanolamine (TEA),
[Bis(2-hydroxyethyl)amino]acetic acid (Bicine), and
N,N-Bis(2-hydroxyethyl)-2-aminoethanesulfonic acid (BES).

4. The pharmaceutical composition according to claim 1, wherein the 2HA comprises or is Tris(hydroxymethyl)aminomethane (Tris) or a pharmaceutically acceptable salt thereof.

5. The pharmaceutical composition according to claim 1, wherein the molar ratio of 2HA:BPA is in the range of 0.5-3, about 0.5, in the range of 0.5-1, about 1, in the range of 1-1.5, in the range of 1-2, about 2, in the range of 2-3, or about 3.

6. The pharmaceutical composition according to any claim 1, wherein the pharmaceutical composition further comprises a polyol, and the molar proportions and/or amounts of the BPA, the 2HA, and the polyol are such that the sum of the molar amounts of BPA and polyol is about equal to or larger than the molar amount of BPA.

7. The pharmaceutical composition according to claim 1, wherein the pharmaceutical composition further comprises a polyol, and the molar proportions and/or amounts of the BPA, the 2HA, and the polyol are such that [2HA]+[polyol]≥0.8×[BPA]; or [2HA]+[polyol]≥0.9×[BPA]; or [2HA]+[polyol]≥1.0×[BPA]; or [2HA]+[polyol]≥1.5×[BPA]; or [2HA]+[polyol]≥2.0×[BPA]; or 0.8×[BPA]≤[2HA]+[polyol]≤5.0×[BPA]; or 0.9×[BPA]≤[2HA]+[polyol]≤4.0×[BPA]; or 1.0×[BPA]≤[2HA]+[polyol]≤3.0×[BPA]; or 1.5×[BPA]≤[2HA]+[polyol]≤2.5×[BPA]; or 2.0×[BPA]≤[2HA]+[polyol]≤2.2×[BPA]; or [2HA]+[polyol]=2.1×[BPA].

8. The pharmaceutical composition according to claim 1, wherein the pharmaceutical composition further comprises at least one of hydrochloric acid, sodium chloride, acetic acid, sodium acetate, polyethylene glycol, a polyol, a saccharide, fructose, mannitol or sorbitol.

9. The pharmaceutical composition according to claim 1, wherein the pharmaceutical composition further comprises a polyol, wherein the polyol is mannitol.

10. The pharmaceutical composition according to claim 1, wherein the pharmaceutical composition comprises L-p-boronophenylalanine, Tris(hydroxymethyl)aminomethane (Tris), and mannitol, and wherein the molar amounts of the L-p-boronophenylalanine, Tris(hydroxymethyl)aminomethane and mannitol are such that [Tris]+[mannitol]>[L-p-boronophenylalanine]; or [Tris]+[mannitol]>1.5×[L-p-boronophenylalanine]; or [Tris]+[mannitol]>2×[L-p-boronophenylalanine]; or [Tris]+[mannitol]<3×[L-p-boronophenylalanine]; or [Tris]+[mannitol]<2.5×[L-p-boronophenylalanine]; or [Tris]+[mannitol]<2×[L-p-boronophenylalanine]; or [Tris]+[mannitol]≥0.8×[BPA]; or [Tris]+[mannitol]≥0.9×[BPA]; or [Tris]+[mannitol]≥1.0×[BPA]; or [Tris]+[mannitol]≥1.5×[BPA]; or [Tris]+[mannitol]≥2.0×[BPA]; or 0.8×[BPA]≤[Tris]+[mannitol]≤5.0×[BPA]; or 0.9×[BPA]≤[Tris]+[mannitol]≤4.0×[BPA]; or 1.0×[BPA]≤[Tris]+[mannitol]≤3.0×[BPA]; or 1.5×[BPA]≤[Tris]+[mannitol]≤2.5×[BPA]; or 2.0×[BPA]≤[Tris]+[mannitol]≤2.2×[BPA]; or [Tris]+[mannitol]=2.1×[BPA].

11. The pharmaceutical composition according to claim 1, wherein the pharmaceutical composition is an aqueous solution, and wherein the aqueous solution optionally has a pH in the range of 6.5 to 8.5.

12. The pharmaceutical composition according to claim 11, wherein the aqueous solution has a pH of 7 to 8, 7.3 to 7.5, 7.35 to 7.45, about pH 7.4, or a physiological pH or a substantially physiological pH.

13. The pharmaceutical composition according to claim 11, wherein the concentration of BPA in the aqueous solution is at least 30 g/L.

14. (canceled)

15. The pharmaceutical composition according to claim 1, wherein the pharmaceutical composition is a dry formulation, wherein the dry formulation is optionally capable of forming an aqueous solution having a pH in the range of 6.5 to 8.5 upon addition of water.

16. (canceled)

17. The pharmaceutical composition according to claim 1, wherein the BPA has at least 30%, at least 40%, at least 50%, at least 60%, at least 70%, at least 80%, at least 90%, at least 95%, at least 97%, at least 98%, or at least 99% boron-10 atoms of all boron atoms in the BPA.

18. (canceled)

19. A method for preparing the pharmaceutical composition according to claim 1, the method comprising
mixing BPA with the 2HA to form an aqueous solution of the BPA and the 2HA,
optionally mixing a polyol with the BPA and the 2HA or adding a polyol to the aqueous solution, and
adjusting the pH of the aqueous solution to a pH in the range of 6.5 to 8.5.

20. The method according to claim 19, the method comprising
mixing L-p-boronophenylalanine and Tris(hydroxymethyl)aminomethane (Tris), to form an aqueous solution, mixing mannitol with the BPA and the 2HA or adding mannitol to the aqueous solution,
optionally adding an agent for adjusting the pH, such as an alkali, to the aqueous solution to completely dissolve the BPA, and
adjusting the pH of the aqueous solution with an acid to a pH of about pH 7.4 or a physiological pH or a substantially physiological pH.

21. The method according to claim 19, wherein the molar ratio of 2HA:BPA is in the range of 0.5-3, about 0.5, in the range of 0.5-1, about 1, in the range of 1-1.5, in the range of 1-2, about 2, in the range of 2-3, or about 3.

22. The method according to claim 19, wherein the molar ratio of Tris(hydroxymethyl)aminomethane (Tris):L-p-boronophenylalanine is in the range of 0.5-3, about 0.5, in the range of 0.5-1, about 1, in the range of 1-1.5, in the range of 1-2, about 2, in the range of 2-3, or about 3.

23. (canceled)

24. The method according to claim 19, wherein the method further comprises drying the aqueous solution, thereby obtaining a dry formulation of the pharmaceutical composition.

25. A method for producing the pharmaceutical composition according to claim 1, wherein the method comprises
providing the pharmaceutical composition as a dry formulation, and
mixing the pharmaceutical composition as the dry formulation with water and optionally with one or more compounds selected from hydrochloric acid, sodium chloride, acetic acid, sodium acetate, polyethylene glycol, a polyol, a saccharide, fructose, mannitol and sorbitol, thereby obtaining the pharmaceutical composition as an aqueous solution.

26. The method according to claim 25, wherein the concentration of the BPA in the aqueous solution is in the range of 30-120 g/L, and the pH of the aqueous solution is about pH 7.4, or a physiological pH or a substantially physiological pH.

27. Use of a 2-hydroxy amine compound (2HA) or a pharmaceutically acceptable salt thereof as defined in claim 1 in dissolving boronophenylalanine (BPA) or a pharmaceutically acceptable salt thereof, thereby forming a pharmaceutical composition comprising BPA as an aqueous solution.
